(19) United States
(12) Patent Application Publication
(10) Pub. No.: US 2024/0327359 A1
(43) Pub. Date: Oct. 3, 2024

(54) 3-ISOXAZOLIDINONE COMPOUND, PREPARATION METHOD, HERBICIDAL COMPOSITION AND APPLICATION THEREOF

(71) Applicant: QINGDAO KINGAGROOT CHEMICAL COMPOUND CO., LTD., QINGDAO, SHANDONG (CN)

(72) Inventors: Lei LIAN, QINGDAO, SHANDONG (CN); Rongbao HUA, QINGDAO, SHANDONG (CN); Xuegang PENG, QINGDAO, SHANDONG (CN)

(21) Appl. No.: 18/290,758

(22) PCT Filed: Aug. 2, 2022
(86) PCT No.: PCT/CN2022/109677

§ 371 Date: Jan. 20, 2024

(30) Foreign Application Priority Data
Aug. 18, 2021 (CN) 202110946153.5
Sep. 4, 2021 (CN) 202111034823.2
Dec. 31, 2021 (CN) 202111677946.8

(51) Int. Cl. C07D 261/04; A01N 25/32; A01N 33/18; A01N 37/18; A01N 37/22; A01N 37/26; A01N 37/28; A01N 37/34; A01N 37/38; A01N 37/40; A01N 43/08; A01N 43/10; A01N 43/36; A01N 43/42; A01N 43/50; A01N 43/54; A01N 43/56; A01N 43/60; A01N 43/653; A01N 43/66; A01N 43/70; A01N 43/707; A01N 43/76; A01N 43/78; A01N 43/80; A01N 43/82; A01N 43/84; A01N 43/86; A01N 43/88; A01N 47/12; A01N 47/16; A01N 47/30; A01N 47/36; A01N 47/38; A01N 57/14; A01P 13/00

(52) U.S. Cl. CPC C07D 261/04; A01N 25/32; A01N 33/18; A01N 37/18; A01N 37/22; A01N 37/26; A01N 37/28; A01N 37/34; A01N 37/38; A01N 37/40; A01N 43/08; A01N 43/10; A01N 43/36; A01N 43/42; A01N 43/50; A01N 43/54; A01N 43/56; A01N 43/60; A01N 43/653; A01N 43/66; A01N 43/70; A01N 43/707; A01N 43/76; A01N 43/78; A01N 43/80; A01N 43/82; A01N 43/84; A01N 43/86; A01N 43/88; A01N 47/12; A01N 47/16; A01N 47/30; A01N 47/36; A01N 47/38; A01N 57/14; A01P 13/00

(57) ABSTRACT

The invention relates to the technical field of pesticides, and in particular to a type of 3-isoxazolidinone compound, as well as a preparation method, a herbicidal composition and an application thereof. The 3-isoxazolidinone compound is as shown in Formula I:

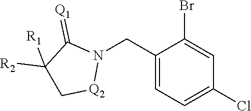

wherein, Q₁ and Q₂ each independently represent O or S; R₁ and R₂ each independently represent alkyl. The present compounds have superior crop safety while maintaining comparable or even better herbicidal activity, thus they are more selective in crops. Moreover, the compounds of the present invention have better anti-volatility to avoid drift.

### TECHNICAL FIELD

The invention relates to the technical field of pesticides, and in particular to a type of 3-isoxazolidinone compound, as well as a preparation method, a herbicidal composition and an application thereof.

### TECHNICAL BACKGROUND

Weed control is one of the most important links in the course of achieving high-efficiency agriculture. Various herbicides are available in the market. For example, U.S. Pat. No. 1,105,357 etc. disclose the use of the compound of general formula

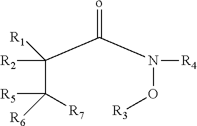

as a herbicide. However, the herbicidal properties of these known compounds against harmful plants and their selectivity to crops are not completely satisfactory. And in recent years, injuries caused by herbicide drifting have become increasingly severe, so it is necessary to pay more attention. For example, hazards of herbicide drift often occur in clomazone, because of its high volatility, and droplet or vapor drift to adjacent areas may cause leaves of certain plants to turn white or yellow. So, scientists still need to do continuous research and develop new herbicides with high efficacy, safety, economy efficiency and different modes of action.

### INVENTION CONTENTS

In order to solve the above-mentioned problems existing in the prior art, the invention provides a type of 3-isoxazolidinone compound, as well as a preparation method, a herbicidal composition and an application thereof. The present compounds have superior crop safety while maintaining comparable or even better herbicidal activity, thus they are more selective in crops. Moreover, the compounds of the present invention have better anti-volatility to avoid drift.

The technical solution adopted by the invention is as follows:

A 3-isoxazolidinone compound, as shown in Formula I:

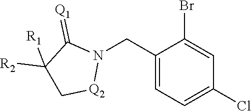

wherein, Q₁ and Q₂ each independently represent O or S;
R₁ and R₂ each independently represent alkyl, preferably C1-C8 alkyl, more preferably C1-C6 alkyl.

In a specific embodiment, Q₁ and Q₂ each independently represent 0; R₁ and R₂ each independently represent methyl (i.e. Compound 1).

In the definition of the compound represented by the above Formula and all of the following structural formulas, the technical terms, whether used alone or used in compound words, represent the following substituents: an alkyl having more than two carbon atoms which may be linear or branched. The alkyl is, for example, C₁ alkyl: methyl; C₂ alkyl: ethyl; C₃ alkyl: propyl such as n-propyl or isopropyl; C₄ alkyl: butyl such as n-butyl, isobutyl, tert-butyl or 2-butyl; C₅ alkyl: pentyl such as n-pentyl; C₆ alkyl: hexyl such as n-hexyl, isohexyl and 1,3-dimethylbutyl. Halogen is fluorine, chlorine, bromine or iodine.

The present invention also provides a method for preparing the 3-isoxazolidinone compound, comprising the following steps:
subjecting the compound of the general formula II and the compound of the general formula III to substitution reaction to obtain the compound of the general formula I, and the chemical reaction equation is as follows:

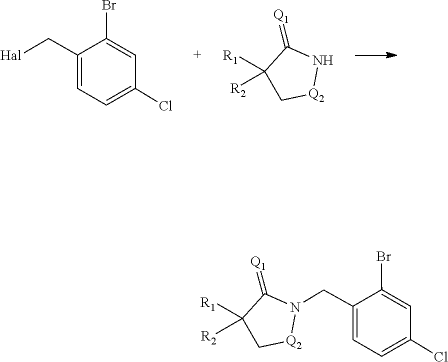

wherein, Hal represents halogen, preferably bromine.

Preferably, the reaction is carried out in the presence of a base and a solvent.

The base is selected from at least one of inorganic bases (such as K₂CO₃, Na₂CO₃, Cs₂CO₃, NaHCO₃, KF, CsF, KOAc, AcONa, K₃PO₄, t-BuONa, EtONa, NaOH, KOH, NaOMe, etc.) or organic bases (such as pyrazole, triethylamine, DIEA, etc.).

The solvent is selected from at least one of DMF, DMA, methanol, ethanol, acetonitrile, dichloroethane, DMSO, Dioxane, dichloromethane, toluene and ethyl acetate.

The present invention also provides a herbicidal composition, comprising (i) a herbicidally effective amount of at least one 3-isoxazolidinone compound;
optionally, the herbicidal composition further comprising (ii) a herbicidally effective amount of one or more additional herbicides and/or safeners;
preferably, the herbicidal composition further comprising (iii) an agrochemically acceptable formulation auxiliary.

The present invention also provides a method for controlling an undesirable plant, comprising applying at least one 3-isoxazolidinone compound or the herbicidal composition in a herbicidally effective amount on a plant, or in its area, or to soil or water to control the emergence or growth of an undesirable plant.

Preferably, the undesirable plant includes herbicide-resistant or -tolerant weed species.

The present invention also provides a use of at least one 3-isoxazolidinone compound or the herbicidal composition for controlling an undesirable plant.

Preferably, the 3-isoxazolidinone compound is used to control a weed among a useful crop.

More preferably, the useful crop includes a transgenic crop or a crop treated by genome editing techniques, and the weed includes herbicide-resistant or -tolerant weed species.

The compounds of the formula I according to the invention have an outstanding herbicidal activity against a broad spectrum of economically important monocotyledonous and dicotyledonous harmful plants. The active compounds of the present invention also act efficiently on perennial weeds which grow from rhizomes, root stocks or other perennial organs and which are difficult to control. In this context, it is generally immaterial whether the substances are applied pre-sowing, pre-emergence or post-emergence. Specifically, examples may be mentioned of some representatives of the monocotyledonous and dicotyledonous weed florae which can be controlled by the compounds according to the invention, without these being a restriction to certain species. Examples of weed species on which the active compounds act efficiently include the monocotyledons, Avena, Lolium, Alopecurus, Phalaris, Echinochloa, Digitaria, Setaria and also Cyperus species from amongst the annual sector, and from amongst the perennial species, Agropyron, Cynodon, Imperata and Sorghum, and also perennial Cyperus species.

In the case of the dicotyledonous weed species, the spectrum of action extends to species such as Galium, Viola, Veronica, Lamium, Stellaria, Amaranthus, Sinapis, Ipomoea, Sida, Matricaria, and Abutilon from amongst the annuals, and Convolvulus, Cirsium, Rumex and Artemisia in the case of the perennial weeds. The active compounds according to the invention also perform outstanding control of harmful plants which occur under the specific conditions of rice growing such as Echinochloa, Sagittaria, Alisma, Eleocharis, Scirpus and Cyperus. If the compounds according to the invention are applied to the soil surface prior to germination, then the weed seedlings are either prevented completely from emerging, or the weeds stop growing when reaching the cotyledon stage and eventually died completely after three to four weeks. In particular, the compounds according to the invention exhibit excellent activity against Apera spica venti, Matsumurella chinense, Fallopia convolvulus, Stellaria media, Veronica hederifolia, Veronica persica, Viola tricolor, Amaranthus, Galium and Kochia.

Although the compounds according to the invention have excellent herbicidal activity against monocotyledonous and dicotyledonous weeds, crop plants of economic importance such as peanut, wheat, barley, rye, rice, corn, sugarbeet, cotton and soya are not damaged at all, or only to a negligible extent. In particular, they have excellent compatibility with cereals such as wheat, barley and corn, in particular wheat. For these reasons, the present compounds are highly suitable for selectively controlling undesired plant growth in plantings for agricultural use or in plantings of ornamentals.

Owing to their herbicidal properties, these active compounds can be employed for controlling harmful plants in plantings of genetically engineered plants that are known or to be introduced. Transgenic plants generally have particularly advantageous properties, for example resistance to certain pesticides, in particular to certain herbicides; resistance to plant diseases or pathogenic microorganisms of plant diseases such as certain insects or microorganisms such as fungi, bacteria or viruses. Other particular traits relate, for example, to the quantity, quality, storage-stability, composition, and to specific ingredients of the product. Thus, transgenic plant products having an increased starch content or modified quality of the starch or a different fatty acid composition are known.

The use of the compounds of the formula I according to the invention or their salts in plantings of economically important transgenic crops and ornamental plants, for example cereals such as wheat, barley, rye, oats, millet, rice, manioc, and corn, or else in plantings of sugarbeet, cotton, soya, rapeseed, potato, tomato, pea, and other vegetable species is preferred. The compounds of the formula I can preferably be used as herbicides in plantings of useful plants which are resistant, or which have been made resistant by genetic engineering to the phytotoxic effects of the herbicides.

Conventional ways for preparing novel plants which have modified traits compared to known plants comprise, for example, traditional breeding methods and the generation of mutants. Alternatively, novel plants having modified traits can be generated with the aid of genetic engineering methods (see, for example, EP-0 221 044A, EP-0 131 624A). For example, there have been described several cases of:
genetically engineered changes in crop plants in order to modify the starch synthesized in the plants (for example, WO 92/11376, WO 92/14827, WO 91/19806);
transgenic crop plants which are resistant to certain herbicides including the glufosinate-P (for example, EP-0 242 236A, EP-0 242 246A), or glyphosates (WO 92/00377), or sulfonylureas (EP-0 257 993A, U.S. Pat. No. 5,013,659A),
transgenic crop plants, for example cotton, having the ability to produce Bacillus thuringiensis toxins (Bt toxins) which impart resistance to certain pests to the plants (EP-0 142 924A, EP-0 193 259A),
transgenic crop plants having a modified fatty acid composition (WO 91/13972).

Numerous molecular biology techniques which allow the preparation of novel transgenic plants having modified traits are known in principle (see, for example, Sambrook et al., 1989, Molecular Cloning, A Laboratory Manual, 2^{nd} ed. Cold Spring Harbor Laboratory Press, Cold Spring Harbor, N.Y.; or Winnacker, Gene und Klone [Genes and Clones], VCH Weinheim, 2^{nd} edition, 1996; or Christou, Trends in Plant Science, 1 (1996) 423-431). In order to carry out such genetic engineering manipulations, it is possible to introduce nucleic acid molecules into plasmids which allow a mutagenesis or a change in the sequence to occur by recombination of DNA sequences. Using the abovementioned standard processes, it is possible, for example, to exchange bases, to remove partial sequences or to add natural or synthetic sequences. To link the DNA fragments with each other, it is possible to attach adaptors or linkers to the fragments.

Plant cells having gene products of reduced activity can be prepared by the following methods, for example, by expressing at least one appropriate antisense-RNA and one sense-RNA to achieve a cosuppression effect, or by expressing at least one appropriately constructed ribozyme which specifically cleaves transcripts of the above-mentioned gene products.

To this end, it is possible to employ both DNA molecules which comprise the entire coding sequence of a gene product including any flanking sequences that may be present, and DNA molecules which comprise only parts of the coding sequence, it being necessary for these parts to be long enough to cause an antisense effect in the cells. It is also possible to use DNA sequences which have a high degree of homology but are not entirely identical to the coding sequences of a gene product.

When expressing nucleic acid molecules in plants, the synthesized protein can be localized in any desired compartment of the plant cells. However, to achieve localization in a certain compartment, it is, for example, possible to link the coding region with DNA sequences which ensure localization in a certain compartment. Such sequences are known to a person skilled in the art (see, for example, Braun et al., EMBO J. 11 (1992), 3219-3227; Wolter et al., Proc. Natl. Acad. Sci. USA 85 (1988), 846-850; Sonnewald et al., Plant J. 1 (1991), 95-106).

The transgenic plant cells can be regenerated into whole plants using known techniques. The transgenic plants can in principle be of any desired plant species, i.e. both monocotyledonous and dicotyledonous plants. In this manner, it is possible to obtain transgenic plants which have modified traits by overexpression, suppression or inhibition of homologous (=natural) genes or gene sequences or by expression of heterologous (=foreign) genes or gene sequences.

When using the active compounds according to the invention in transgenic crops, in addition to the effects against harmful plants which can be observed in other crops, there are frequently effects which are specific for the application in the respective transgenic crop, for example a modified or broadened spectrum of weeds which can be controlled, a modified application amount which can be used for the application, preferably good combinability with the transgenic crops which are resistant to the herbicides, and an effect on the growth and the yield of the transgenic crop plants. The invention therefore also provides for a use of the compounds according to the invention as herbicides for controlling harmful plants among transgenic crop plants.

In addition, the substances according to the invention have outstanding growth-regulating properties in crop plants. They engage in the plant metabolism in a regulating manner and can this be employed for the targeted control of plant constituents and for facilitating harvesting, for example by provoking desiccation and stunted growth. Furthermore, they are also suitable for generally regulating and inhibiting undesirable plant growth, without destroying the growth of crop plants. Inhibition of plant growth plays an important role in many monocotyledonous and dicotyledonous crops because lodging can be reduced hereby, or prevented completely.

The compounds according to the invention can be applied in the customary formulations in the form of wettable powders, emulsifiable concentrates, sprayable solutions, dusts or granules. The invention therefore also provides herbicidal compositions comprising the compounds of the formula I. The compounds of the formula I can be formulated in various ways depending on the prevailing biological and/or chemico-physical parameters. Examples of suitable formulation options are: wettable powders (WP), water-soluble powders (SP), water-soluble concentrates, emulsifiable concentrates (EC), emulsions (EW) such as oil-in-water and water-in-oil emulsions, sprayable solutions, suspension concentrates (SC), oil dispersions (OD), oil- or water-based dispersions, oil-miscible solutions, dust powders (DP), capsule suspensions (CS), seed-dressing compositions, granules for broadcasting and soil application, spray granules, coating granules and adsorption granules, water-dispersible granules (WG), water-soluble granules (SG), ULV formulations, microcapsules and waxes. These individual formulation types are known in principle and are described, for example, in Winnacker-Küchler, “Chemische Technologie” [Chemical Technology], Volume 7, C. Hauser Verlag Munich, 4^{th} Edition, 1986; Wade van Valkenburg, “Pesticide Formulations”, Marcel Dekker, N.Y., 1973; K. Martens, “Spray Drying” Handbook, 3^{rd} Ed. 1979, G. Goodwin Ltd. London.

The necessary formulation auxiliaries such as inert materials, surfactants, solvents and other additives are likewise known and are described, for example, in Watkins, “Handbook of Insecticide Dust Diluents and Carriers”, 2^{nd} Ed., Dorland Books, Caldwell N.J.; H. v. Olphen, “An Introduction to Clay Colloid Chemistry”, 2^{nd} Ed., J. Wiley & Sons, N.Y.; C. Marsden, “Solvents Guide”, 2^{nd} Ed., Interscience, N.Y. 1963; “McCutcheon's Detergents and Emulsifiers Annual”, MC Publ. Corp., Ridgewood N.J.; Sisley and Wood, “Encyclopedia of Surface Active Agents”, Chem. Publ. Co. Inc., N.Y., 1964; Schonfeldt, “Grenzflichenaktive Äthylenoxidaddkte” [Surface-active ethylene oxide adducts], Wiss. Verlagagesell., Stuttgart, 1976; Winnacker-Kuchler, “Chemische Technologie” [Chemical Technology], Volume 7, C. Hauser Verlag Munich, 4^{th} Edition, 1986.

Wettable powders are preparations which are uniformly dispersible in water and which contain, in addition to the active compound, a diluent or inert substance, surfactants of ionic and/or nonionic type (wetting agents, dispersants), for example polyethoxylated alkyl phenols, polyethoxylated fatty alcohols, polyethoxylated fatty amines, fatty alcohol polyglycol ether sulfates, alkanesulfonates, alkylphenylsulfonates, sodium lignosulfonate, sodium 2,2′-dinaphthylmethane-6,6′-disulfonate, sodium dibutylnaphthalenesulfonate or else sodium methyl oleoyl taurate. To prepare the wettable powders, the herbicidally active compounds are finely ground, for example in customary apparatus such as hammer mills, fan mills and air-jet mills, and are mixed simultaneously or subsequently with the formulation auxiliaries.

Emulsifiable concentrates are prepared by dissolving the active compound in an organic solvent, for example butanol, cyclohexanone, dimethylformamide, xylene or else relatively high-boiling aromatic compounds or hydrocarbons or mixtures of the solvents, with the addition of one or more surfactants of ionic and/or nonionic type (emulsifiers). Examples of emulsifiers which can be used are calcium alkylarylsulfonates such as Ca dodecylbenzenesulfonate, or nonionic emulsifiers such as fatty acid polyglycol esters, alkylaryl polyglycol ethers, fatty alcohol polyglycol ethers, propylene oxide-ethylene oxide condensation products, alkyl polyethers, sorbitan esters, for example sorbitan fatty acid esters, or polyoxyethylene sorbitan esters, for example polyoxyethylene sorbitan fatty acid esters.

Dusts are obtained by grinding the active compound with finely divided solid substances, for example talc, natural clays such as kaolin, bentonite and pyrophyllite, or diatomaceous earth. Water- or oil-based suspension concentrates can be prepared, for example, by wet milling using commercially customary bead mills, with or without the addition of surfactants as already mentioned above, for example, in the case of the other formulation types.

Emulsions, for example oil-in-water emulsions (EW), can be prepared by means of stirrers, colloid mills and/or static mixers using aqueous organic solvents and, if desired, surfactants as already mentioned above, for example, in the case of the other formulation types.

Granules can be prepared either by spraying the active compound onto adsorptive, granulated inert material, or by applying active-compound concentrates to the surface of carriers such as sand, kaolinites or granulated inert material by means of adhesive binders, for example polyvinyl alcohol, sodium polyacrylate or else mineral oils. Suitable active compounds can also be granulated in the manner which is customary for the preparation of fertilizer granules, if desired in a mixture with fertilizers. Water-dispersible granules are generally prepared by the customary processes such as spray-drying, fluidized-bed granulation, disk granulation, mixing using high-speed mixers, and extrusion without solid inert material.

For the preparation methods of granules by disk, fluidized-bed, extruder and spray see, for example processes in “Spray-Drying Handbook” 3^{rd} ed. 1979, G. Goodwin Ltd., London; J. E. Browning, “Agglomeration”, Chemical and Engineering, 1967, pages 147 ff.; “Perry's Chemical Engineer's Handbook”, 5^{th} Ed., McGraw-Hill, New York 1973, pp. 8-57. For further details on the formulation of crop protection products, see, for example G. C. Klingman, “Weed Control as a Science”, John Wiley and Sons Inc., New York, 1961, pages 81-96 and J. D. Freyer, S. A. Evans, “Weed Control Handbook”, 5^{th} Ed., Blackwell Scientific Publications, Oxford, 1968, pages 101-103.

The agrochemical formulations generally contain from 0.1 to 99% by weight, in particular from 0.1 to 95% by weight, of the active compound of the formula I. In wettable powders the concentration of the active compound is, for example, from about 10 to 99% by weight, the remainder to 100% by weight consisting of customary formulation constituents. In emulsifiable concentrates the concentration of the active compound can be from about 1 to 90%, preferably from 5 to 80%, by weight. Formulations in the form of dusts contain from 1 to 30% by weight of the active compound, preferably from 5 to 20% by weight of the active compound, while sprayable solutions contain from about 0.05 to 80%, preferably from 2 to 50%, by weight of the active compound. In the case of water-dispersible granules the content of active compound depends partly on whether the active compound is in liquid or solid form and on auxiliaries, fillers, etc. that are used in granulation. In water-dispersible granules the content of the active compound, for example, is between 1 and 95% by weight, preferably between 10 and 80% by weight.

In addition, the formulations of the active compound may comprise tackifiers, wetting agents, dispersants, emulsifiers, penetrants, preservatives, antifreeze agents, solvents, fillers, carriers, colorants, defoamers, evaporation inhibitors and pH and viscosity regulators which are customary in each case.

Based on these formulations it is also possible to produce combinations with other pesticidally active substances, for example insecticides, acaricides, herbicides and fungicides, and also with safeners, fertilizers and/or growth regulators, in the form of a pre-mix or tank mix.

Suitable active compounds which can be combined with the compounds of formula I according to the invention in mixed formulations or in a tank mix are the known active compounds as described in, for example World Herbicide New Product Technology Handbook, China Agricultural Science and Farming Techniques Press, 2010.9 and in the literature cited therein. For example, the following active compounds may be mentioned as herbicides which can be combined with the Compound 1 (note: the compounds are either named by the “common name” in accordance with the International Organization for Standardization (ISO) or by the chemical names, if appropriate together with a customary code number):
(1) HPPD (Hydroxyphenyl Pyruvate Dioxygenase) inhibitors: including but not limited to the following types,
1) the triketones, e.g. sulcotrione (CAS NO: 99105-77-8); mesotrione (CAS NO: 104206-82-8); bicyclopyrone (CAS NO: 352010-68-5); tembotrione (CAS NO: 335104-84-2); tefuryltrione (CAS NO: 473278-76-1); and benzobicyclon (CAS NO: 156963-66-5);
2) the diketonitriles, e.g. 2-cyano-3-cyclopropyl-1-(2-methylsulphonyl-4-trifluoromethylphenyl)-propane-1,3-dione (CAS NO: 143701-75-1); 2-cyano-3-cyclopropyl-1-(2-methylsulphonyl-3,4-dichlorophenyl)-propane-1,3-dione (CAS NO: 212829-55-5); and 2-cyano-1-[4-(methylsulphonyl)-2-trifluoromethylphenyl]-3-(1-methylcyclopropyl)propane-1,3-dione (CAS NO: 143659-52-3);
3) the isoxazoles, e.g. isoxaflutole (CAS NO: 141112-29-0); isoxachlortole (CAS NO: 141112-06-3); and clomazone (CAS NO: 81777-89-1);
4) the pyrazoles, e.g. topramezone (CAS NO: 210631-68-8); pyrasulfotole (CAS NO: 365400-11-9); pyrazoxyfen (CAS NO: 71561-11-0); pyrazolate (CAS NO: 58011-68-0); benzofenap (CAS NO: 82692-44-2); bipyrazone (CAS NO: 1622908-18-2); tolpyralate (CAS NO: 1101132-67-5); fenpyrazone (CAS NO: 1992017-55-6); cypyrafluone (CAS NO: 1855929-45-1); and tripyrasulfone (CAS NO: 1911613-97-2);
5) the benzophenones;
6) others: lancotrione (CAS NO: 1486617-21-3); fenquinotrione (CAS NO: 1342891-70-6); and flusulfinam (CAS NO: 2421252-30-2);
and those mentioned in patent CN105264069A.
(2) EPSPS (Enolpyruvyl Shikimate Phosphate Synthase) inhibitors: e.g. sulphosate, glyphosate, glyphosate-isopropylammonium, and glyphosate-trimesium;
(3) PPO (Protoporphyrinogen Oxidase) inhibitors: classified into pyrimidinediones, diphenyl-ethers, phenylpyrazoles, N-phenylphthalimides, thiadiazoles, oxadiazoles, triazolinones, oxazolidinediones, and other herbicides with different chemical structures.

In an exemplary embodiment, the pyrimidinedione herbicides include but are not limited to, butafenacil (CAS NO: 134605-64-4), saflufenacil (CAS NO: 372137-35-4), benzfendizone (CAS NO: 158755-95-4), tiafenacil (CAS NO: 1220411-29-9), [3-[2-chloro-4-fluoro-5-(1-methyl-6-trifluoromethyl-2,4-dioxo-1,2,3,4-tetrahydropyrimidin-3-yl)phenoxy]-2-pyridyloxy]acetic acid ethyl ester (Epyrifenacil, CAS NO: 353292-31-6), 1-methyl-6-trifluoromethyl-3-(2,2,7-trifluoro-3-oxo-4-prop-2-ynyl-3,4-dihydro-2H-benzo[1,4]oxazin-6-yl)-1H-pyrimidine-2,4-dione (CAS NO: 1304113-05-0), 3-[7-chloro-5-fluoro-2-(trifluoromethyl)-1H-benzimidazol-4-yl]-1-methyl-6-(trifluoromethyl)-1H-pyrimidine-2,4-dione (CAS NO: 212754-02-4), flupropacil (CAS NO: 120890-70-2), isoxazoline-containing uracils disclosed in CN105753853A (e.g. compound

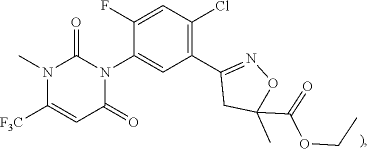 [SMILES: CCOC(=O)C1(C)CC(c2cc(-n3c(=O)cc(C(F)(F)F)n(C)c3=O)c(F)cc2Cl)=NO1]

uracil pyridines disclosed in WO2017/202768, and uracils disclosed in WO2018/019842;
the diphenyl-ether herbicides include but are not limited to, fomesafen (CAS NO: 72178-02-0), oxyfluorfen (CAS NO: 42874-03-3), aclonifen (CAS NO: 74070-46-5), ethoxyfen-ethyl (CAS NO: 131086-42-5), lactofen (CAS NO: 77501-63-4), chlomethoxyfen (CAS NO: 32861-85-1), chlomitrofen (CAS NO: 1836-77-7), fluoroglycofen-ethyl (CAS NO: 77501-90-7), acifluorfen or its sodium salt (acifluorfen, CAS NO: 50594-66-6 or 62476-59-9), bifenox (CAS NO: 42576-02-3), ethoxyfen (CAS NO: 188634-90-4), fluoronitrofen (CAS NO: 13738-63-1), furyloxyfen (CAS NO: 80020-41-3), nitrofluorfen (CAS NO: 42874-01-1), and halosafen (CAS NO: 77227-69-1);
the phenylpyrazole herbicides include but are not limited to, pyraflufen-ethyl (CAS NO: 129630-19-9), and fluazolate (CAS NO: 174514-07-9);
the N-phenylphthalimide herbicides include but are not limited to, flumioxazin (CAS NO: 103361-09-7), cinidon-ethyl (CAS NO: 142891-20-1), glumipropyn (CAS NO: 84478-52-4), and flumiclorac-pentyl (CAS NO: 87546-18-7);
the thiadiazole herbicides include but are not limited to, fluthiacet-methyl (CAS NO: 117337-19-6), fluthiacet (CAS NO: 149253-65-6), and thidiazimin (CAS NO: 123249-43-4);
the oxadiazole herbicides include but are not limited to, ovarialgia (CAS NO: 39807-15-3), and oxadiazon (CAS NO: 19666-30-9);
the triazolinone herbicides include but are not limited to, carfentrazone (CAS NO: 128621-72-7), carfentrazone-ethyl (CAS NO: 128639-02-1), sulfentrazone (CAS NO: 122836-35-5), azafenidin (CAS NO: 68049-83-2), and bencarbazone (CAS NO: 173980-17-1);
the oxazolidinedione herbicides include but are not limited to, pentoxazone (CAS NO: 110956-75-7);
the other herbicides include but are not limited to, pyraclonil (CAS NO: 158353-15-2), flufenpyr-ethyl (CAS NO: 188489-07-8), profluazol (CAS NO: 190314-43-3), trifludimoxazin (CAS NO: 1258836-72-4), N-ethyl-3-(2,6-dichloro-4-trifluoromethylphenoxy)-5-methyl-1H-pyrazole-1-carboxamide (CAS NO: 452098-92-9), N-tetrahy drofurfuryl-3-(2,6-dichloro-4-trifluoromethylphenoxy)-5-methyl-1H-pyrazole-1-carboxamide (CAS NO: 915396-43-9), N-ethyl-3-(2-chloro-6-fluoro-4-trifluoromethylphenoxy)-5-methyl-1H-pyrazole-1-carboxamide (CAS NO: 452099-05-7), N-tetrahydrofurfuryl-3-(2-chloro-6-fluoro-4-trifluoromethylphenoxy)-5-methyl-1H-pyrazole-1-carboxamide (CAS NO: 452100-03-7), 3-[7-fluoro-3-oxo-4-(prop-2-ynyl)-3,4-dihydro-2H-benzo[1,4]oxazin-6-yl]-1,5-dimethyl-6-thioxo[1,3,5]triazinane-2,4-dione (CAS NO: 451484-50-7), 2-(2,2,7-trifluoro-3-oxo-4-prop-2-ynyl-3,4-dihydro-2H-benzo[1,4]oxazin-6-yl)-4,5,6,7-tetrahydroisoindole-1,3-dione (CAS NO: 1300118-96-0), methyl(E)-4-[2-chloro-5-[4-chloro-5-(difluoromethoxy)-1H-methylpyrazol-3-yl]-4-fluorophenoxy]-3-methoxy-but-2-enoate (CAS NO: 948893-00-3), phenylpyridines disclosed in WO2016/120116, benzoxazinone derivatives disclosed in EP09163242.2, and carboxylic acid derivative-substituted imino aryl compounds disclosed in CN113105405A;
(4) ALS (Acetolactate Synthase) inhibitors include but are not limited to the following herbicides or mixtures thereof:

Sulfonylureas, e.g. amidosulfuron, azimsulfuron, bensulfuron, bensulfuron-methyl, chlorimuron, chlorimuron-ethyl, chlorsulfuron, cinosulfuron, cyclosulfamuron, ethametsulfuron, ethametsulfuron-methyl, ethoxysulfuron, flazasulfuron, flucetosulfuron, flupyrsulfuron, flupyrsulfuron-methyl-sodium, foramsulfuron, halosulfuron, halosulfuron-methyl, imazosulfuron, iodosulfuron, iodosulfuron-methyl-sodium, iofensulfuron, iofensulfuron-sodium, mesosulfuron, metazosulfuron, metsulfuron, metsulfuron-methyl, nicosulfuron, orthosulfamuron, oxasulfuron, primisulfuron, primisulfuron-methyl, propyrisulfuron, prosulfuron, pyrazosulfuron, pyrazosulfuron-ethyl, rimsulfuron, sulfometuron, sulfometuron-methyl, sulfosulfuron, thifensulfuron, thifensulfuron-methyl, triasulfuron, tribenuron, tribenuron-methyl, trifloxysulfuron, trifloxysulfuron-Na, triflusulfuron, triflusulfuron-methyl, and tritosulfuron;

Imidazolinones, e.g. imazamethabenz, imazamethabenz-methyl, imazamox, imazapic, imazapyr, imazaquin, and imazethapyr;

Triazolopyrimidine herbicides and sulfonanilides, e.g. cloransulam, cloransulam-methyl, diclosulam, flumetsulam, florasulam, metosulam, penoxsulam, pyroxsulam, pyrimisulfan, and triafamone;

Pyrimidinyl benzoic acids, e.g. bispyribac, bispyribac-sodium, pyribenzoxim, pyriftalid, pyriminobac, pyriminobac-methyl, pyrithiobac, pyrithiobac-sodium, 4-[[[2-[(4,6-dimethoxy-2-pyrimidinyl)oxy]phenyl]methyl]amino]benzoic acid 1-methylethyl ester (CAS NO: 420138-41-6), 4-[[[2-[(4,6-dimethoxy-2-pyrimidinyl)oxy]phenyl]methyl]amino]benzoic acid propyl ester (CAS NO: 420138-40-5), and N-(4-bromophenyl)-2-[(4,6-dimethoxy-2-pyrimidinyl)oxy]benzenemethanamine (CAS NO: 420138-01-8);

Sulfonylaminocarbonyl-triazolinone herbicides, e.g. flucarbazone, flucarbazone-sodium, propoxycarbazone, propoxycarbazone-sodium, thiencarbazone, and thiencarbazone-methyl.
(5) ACCase (Acetyl CoA Carboxylase) inhibitors: e.g. fenthiaprop, alloxydim, alloxydim-sodium, butroxydim, clethodim, clodinafop, clodinafop-propargyl, cycloxydim, cyhalofop, cyhalofop-butyl, diclofop, diclofop-methyl, fenoxaprop, fenoxaprop-ethyl, fenoxaprop-P, fenoxaprop-P-ethyl, fluazifop, fluazifop-butyl, fluazifop-P, fluazifop-P-butyl, haloxyfop, haloxyfop-methyl, haloxyfop-P, haloxyfop-P-methyl, metamifop, pinoxaden, profoxydim, propaquizafop, quizalofop, quizalofop-ethyl, quizalofop-tefuryl, quizalofop-P, quizalofop-P-ethyl, quizalofop-P-tefuryl, sethoxydim, tepraloxydim, tralkoxydim, 4-(4′-chloro-4-cyclopropyl-2′-fluoro[1,1′-biphenyl]-3-yl)-5-hydroxy-2,2,6,6-tetramethyl-2H-pyran-3(6H)-one (CAS NO: 1312337-72-6); 4-(2′,4′-dichloro-4-cyclopropyl[1,1′-biphenyl]-3-yl)-5-hydroxy-2,2,6,6-tetramethyl-2H-pyran-3(6H)-one (CAS NO: 1312337-45-3); 4-(4′-chloro-4-ethyl-2′-fluoro[1,1′-biphenyl]-3-yl)-5-hydroxy-2,2,6,6-tetramethyl-2H-pyran-3(6H)-one (CAS NO: 1033757-93-5); 4-(2′,4′-dichloro-4-ethyl[1,1′-biphenyl]-3-yl)-2,2,6,6-tetramethyl-2H-pyran-3,5(4H,6H)-dione (CAS NO: 1312340-84-3); 5-(acetyloxy)-4-(4′-chloro-4-cyclopropyl-2′-fluoro[1,1′-biphenyl]-3-yl)-3,6-dihydro-2,2,6,6-tetramethyl-2H-pyran-3-one (CAS NO: 1312337-48-6); 5-(acetyloxy)-4-(2′,4′-dichloro-4-cyclopropyl-[1,1′-biphenyl]-3-yl)-3,6-dihydro-2,2,6,6-tetramethyl-2H-pyran-3-one; 5-(acetyloxy)-4-(4′-chloro-4-ethyl-2′-fluoro[1,1′-biphenyl]-3-yl)-3,6-dihydro-2,2,6,6-tetramethyl-2H-pyran-3-one (CAS NO: 1312340-82-1); 5-(acetyloxy)-4-(2′,4′-dichloro-4-ethyl[1,1′-biphenyl]-3-yl)-3,6-dihydro-2,2,6,6-tetramethyl-2H-pyran-3-one (CAS NO: 1033760-55-2); 4-(4′-chloro-4-cyclopropyl-2′-fluoro[1,1′-biphenyl]-3-yl)-5,6-dihydro-2,2,6,6-tetramethyl-5-oxo-2H-pyran-3-yl methyl carbonate (CAS NO: 1312337-51-1); 4-(2′,4′-dichloro-4-cyclopropyl-[1,1′-biphenyl]-3-yl)-5,6-dihydro-2,2,6,6-tetramethyl-5-oxo-2H-pyran-3-yl methyl carbonate; 4-(4′-chloro-4-ethyl-2′-fluoro[1,1′-biphenyl]-3-yl)-5,6-dihydro-2,2,6,6-tetramethyl-5-oxo-2H-pyran-3-yl methyl carbonate (CAS NO: 1312340-83-2); and 4-(2′,4′-dichloro-4-ethyl[1,1′-biphenyl]-3-yl)-5,6-dihydro-2,2,6,6-tetramethyl-5-oxo-2H-pyran-3-yl methyl carbonate (CAS NO: 1033760-58-5);
(6) GS (Glutamine Synthetase) inhibitors: e.g. bialaphos/bilanafo, bilanaphos-natrium, glufosinate-ammonium, glufosinate, and glufosinate-P;
(7) PDS (Phytoene Desaturase) inhibitors: e.g. flurochloridone, flurtamone, beflubutamid, norflurazon, fluridone, diflufenican, picolinafen, and 4-[3-(trifluoromethyl)phenoxy]-2-[4-(trifluoromethyl)phenyl]pyrimidine (CAS NO: 180608-33-7);
(8) DHPS (Dihydropteroate Synthase) inhibitors: e.g. asulam;
(9) DXPS (Deoxy-D-Xyulose Phosphate Synthase) inhibitors: e.g. bixlozone, and clomazone;
(10) HST (Homogentisate Solanesyltransferase) inhibitors: e.g. cyclopyrimorate;
(11) SPS (Solanesyl Diphosphate Synthase) inhibitors: e.g. aclonifen;
(12) Cellulose Synthesis inhibitors: e.g. indaziflam, triaziflam, chlorthiamid, dichlobenil, isoxaben, flupoxam, 1-cyclohexyl-5-pentafluorophenyloxy-14-[1,2,4,6]thiatriazin-3-ylamine (CAS NO: 175899-01-1), and the azines disclosed in CN109688807A;
(13) VLCFAS (Very Long-Chain Fatty Acid Synthesis) inhibitors: including but not limited to the following types,
1) Chloroacetamides, e.g. acetochlor, alachlor, butachlor, dimethachlor, dimethenamid, dimethenamid-P, metazachlor, metolachlor, S-metolachlor, pethoxamid, pretilachlor, propachlor, propisochlor, and thenylchlor;
2) α-Oxyacetamides, e.g. flufenacet, and mefenacet;
3) α-Thioacetamides, e.g. anilofos, and piperophos;
4) Azolyl-carboxamides, e.g. cafenstrole, fentrazamide, and ipfencarbazone;
5) Benzofuranes, e.g. benfuresate, and ethofumesate;
6) Isoxazolines, e.g. fenoxasulfone, and pyroxasulfone;
7) Ethylene oxides, e.g. indanofan, and tridiphane;
8) Thiocarbamates, e.g. cycloate, dimepiperate, S-ethyl dipropyl(thiocarbamate) (EPTC), esprocarb, molinate, orbencarb, prosulfocarb, thiobencarb/benthiocarb, triallate, vernolate, isoxazolines of formula II.1, II.2, I.3, I.4, I.5, I.6, I.7, II.8 and 11.9, and other isoxazoline compounds mentioned in patents such as WO 2006/024820, WO 2006/037945, WO 2007/071900 and WO 2007/096576.

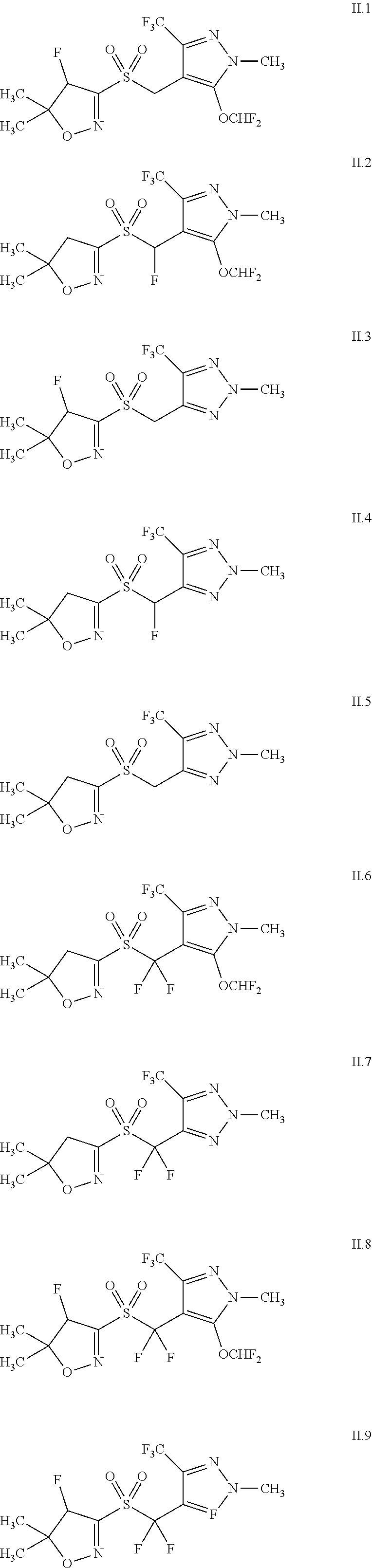

(14) Fatty acid thioesterase inhibitors: e.g. cinmethylin, and methiozolin;
(15) Serine threonine protein phosphatase inhibitors: e.g. endothall;
(16) Lycopene cyclase inhibitors: e.g. amitrole.
(17) Microtubule Assembly inhibitors: e.g. benefin/benfluralin, butralin, dithiopyr, thiazopyr, ethalfluralin, prodiamine, butamifos, oryzalin, pendimethalin, chlorthal-dimethyl/DCPA, DMPA

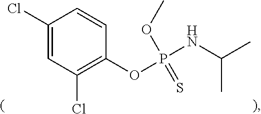 [SMILES: COP(=S)(NC(C)C)Oc1ccc(Cl)cc1Cl]

trifluralin, propyzamide/pronamide, and dinitramine (CAS NO: 29091-05-2);
(18) Auxin Mimics: e.g. 2,4,5-trichlorophenoxyacetic acid (2,4,5-T), 2,4-dichlorophenoxyacetic acid (2,4-D), 2,4-dichlorophenoxybutyric acid (2,4-DB), 3,4-dichlorophenoxybutyric acid (3,4-DB), 2,4-DEB (CAS NO: 94-83-7), 4-chlorophenoxyacetic acid (4-CPA), 4-CPP (CAS NO: 3307-39-9), cloprop (CAS NO: 101-10-0), clofop (CAS NO: 26129-32-8), clomeprop, dichlorprop, (4-chloro-2-methylphenoxy)acetic acid (MCPA), 2-methyl-4-chlorophenoxybutyric acid (MCPB), mecoprop, aminopyralid, clopyralid, aminocyclopyrachlor, florpyrauxifen, halauxifen, quinclorac, benazolin-ethyl, picloram, chloramben, quinmerac, fluroxypyr, triclopyr, and dicamba;
(19) D1 Serine 264 binders (and other non-histidine 215 binders): e.g. ametryne, atrazine, cyanazine, dimethametryn, prometon, prometryne, propazine, simazine, terbuthylazine, terbutryne, chlorotoluron, diuron, fluometuron, isoproturon, linuron, metobromuron, methabenzthiazuron (CAS NO: 18691-97-9), monuron, tebuthiuron, hexazinone, metamitron, metribuzin, bromacil, lenacil, terbacil, chloridazon/pyrazon, amicarbazone, desmedipham, phenmedipham, chloranocryl/dicryl (CAS NO: 2164-09-2), propanil, chlorazine (CAS NO: 580-48-3), cyprazine (CAS NO: 22936-86-3), trietazine (CAS NO: 1912-26-1), methoprotryne (CAS NO: 841-06-5), and simetryn;
(20) D1 Histidine 215 binders: e.g. bromoxynil, ioxynil, pyridate, and bentazon;
(21) Auxin Transport inhibitors: e.g. diflufenzopyr (CAS NO: 109293-97-2), and naptalam;
(22) PS1 Electron Diversion: e.g. diquat, and paraquat;
(23) Inhibition of Microtubule Organization: e.g. carbetamide (CAS NO: 16118-49-3), barban (CAS NO: 101-27-9), and chlorpropham;
(24) Uncouplers: e.g. dinoseb, and 4,6-dinitro-o-cresol (DNOC);
(25) Others: e.g. bensulide, bromobutide, cumyluron, difenzoquat, pyributicarb (CAS NO: 88678-67-5), disodium methylarsenate (DSMA), sodium monomethylarsenate (MSMA), dymron/daimuron, etobenzanid, flamprop-m, fosamine, oxaziclomefone, pelargonic acid, diphenamid, naproanilide, napropamide, napropamide-m, benzofluor (CAS NO: 68672-17-3), cambendichlor (CAS NO: 56141-00-5), dipropalin (CAS NO: 1918-08-7), etnipromid (CAS NO: 76120-02-0), bromobonil (CAS NO: 25671-46-9), monisouron (CAS NO: 55807-46-0), bromofenoxim (CAS NO: 13181-17-4), clacyfos (CAS NO: 215655-76-8), chlorazifop (CAS NO: 60074-25-1), kuicaoxi, brompyrazon (CAS NO: 304284-0), ethiozin, methiuron (CAS NO: 21540-35-2), metoxuron (CAS NO: 19937-59-8), tetrafluron (CAS NO: 27954-37-6), thidiazuron (CAS NO: 51707-55-2), acrolein, and funaihecaoling.

In a specific embodiment, the additional herbicide (component B) combined with Compound 1 (component A) is selected from one or more of the following compounds: mesotrione (CAS NO: 104206-82-8), tefuryltrione (CAS NO: 473278-76-1), benzobicyclon (CAS NO: 156963-66-5), bipyrazone (CAS NO: 1622908-18-2), cypyrafluone (CAS NO: 1855929-45-1), tripyrasulfone (CAS NO: 1911613-97-2), fenpyrazone (CAS NO: 1992017-55-6), fenquinotrione (CAS NO: 1342891-70-6),

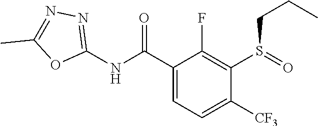 [SMILES: CCC[S@@](=O)c1c(C(F)(F)F)ccc(C(=O)Nc2nnc(C)o2)c1F]

(CAS NO: 2421252-30-2), isoxaflutole (CAS NO: 141112-29-0), flurochloridone (CAS NO: 61213-25-0), diflufenican (CAS NO: 83164-33-4), picolinafen (CAS NO: 137641-05-5), beflubutamid (CAS NO: 113614-08-7), fluridone (CAS NO: 59756-60-4), clomazone (CAS NO: 81777-89-1), bixlozone (CAS NO: 81777-95-9), bensulfuron-methyl (CAS NO: 83055-99-6), pyrazosulfuron-ethyl (CAS NO: 93697-74-6), halosulfuron (CAS NO: 100784-20-1), propyrisulfuron (CAS NO: 570415-88-2), metazosulfuron (CAS NO: 868680-84-6), ethoxysulfuron (CAS NO: 126801-58-9), mesosulfuron-methyl (CAS NO: 208465-21-8), nicosulfuron (CAS NO: 111991-09-4), triafamone (CAS NO: 874195-61-6), penoxsulam (CAS NO: 219714-96-2), imazamox (CAS NO: 114311-32-9), imazethapyr (CAS NO: 81385-77-5), florasulam (CAS NO: 145701-23-1), bispyribac (CAS NO: 125401-92-5), oxyfluorfen (CAS NO: 42874-03-3), fomesafen (CAS NO: 72178-02-0), oxadiazon (CAS NO: 19666-30-9), oxadiargyl (CAS NO: 39807-15-3), sulfentrazone (CAS NO: 122836-35-5), pyraclonil (CAS NO: 158353-15-2), flumioxazin (CAS NO: 103361-09-7), pentoxazone (CAS NO: 110956-75-7), tiafenacil (CAS NO: 1220411-29-9), saflufenacil (CAS NO: 372137-35-4), trifludimoxazin (CAS NO: 1258836-72-4), epyrifenacil (CAS NO: 353292-31-6)

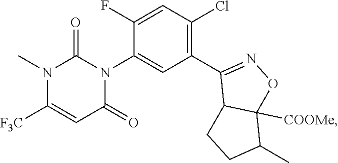 [SMILES: COC(=O)C12ON=C(c3cc(-n4c(=O)cc(C(F)(F)F)n(C)c4=O)c(F)cc3Cl)C1CCC2C]

### (CAS NO: 2669821-71-8),

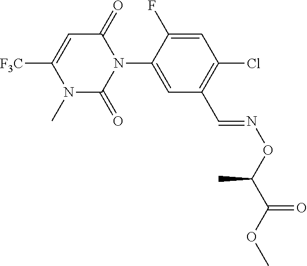 [SMILES: COC(=O)[C@@H](C)O/N=C/c1cc(-n2c(=O)cc(C(F)(F)F)n(C)c2=O)c(F)cc1Cl]

(CAS NO: 2669111-66-2), benmicaozuo (CAS NO: 1949837-17-5), atrazine (CAS NO: 1912-24-9), prometryne (CAS NO: 7287-19-6), simetryn (CAS NO: 1014-70-6), terbutylazine (CAS NO: 5915-41-3), terbutryne (CAS NO: 886-50-0), ametryne (CAS NO: 834-12-8), metribuzin (CAS NO: 21087-64-9), amicarbazone (CAS NO: 129909-90-6), chlorotoluron (CAS NO: 15545-48-9), isoproturon (CAS NO: 34123-59-6), diuron (CAS NO: 330-54-1), propanil (CAS NO: 709-98-8), bentazon (CAS NO: 25057-89-0), bromoxynil octanoate (CAS NO: 1689-99-2), butralin (CAS NO: 33629-47-9), pendimethalin (CAS NO: 40487-42-1), dithiopyr (CAS NO: 97886-45-8), acetochlor (CAS NO: 34256-82-1), butachlor (CAS NO: 23184-66-9), pretilachlor (CAS NO: 51218-49-6), metolachlor (CAS NO: 87392-12-9), mefenacet (CAS NO: 73250-68-7), S-metolachlor (CAS NO: 178961-20-1), flufenacet (CAS NO: 142459-58-3), napropamide (CAS NO: 15299-99-7), pyroxasulfone (CAS NO: 447399-55-5), anilofos (CAS NO: 64249-01-0), dimethenamid (CAS NO: 87674-68-8), fentrazamide (CAS NO: 158237-07-1), ipfencarbazone (CAS NO: 212201-70-2), dimethenamid-P (CAS NO: 163515-14-8), bromobutide (CAS NO: 74712-19-9), prosulfocarb (CAS NO: 52888-80-9), molinate (CAS NO: 2212-67-1), quinclorac (CAS NO: 84087-01-4), fluroxypyr (CAS NO: 69377-81-7), fluroxypyr-meptyl (CAS NO: 81406-37-3), halauxifen (CAS NO: 943831-98-9), dicamba (CAS NO: 1918-00-9), florpyrauxifen (CAS NO: 1390661-72-9), (4-chloro-2-methylphenoxy)acetic acid (CAS NO: 94-74-6), 2-methyl-4-chlorophenoxymonothioacetic acid S-ethyl ester (CAS NO: 25319-90-8), 2-methyl-4-chlorophenoxyacetic acid sodium salt (CAS NO: 3653-48-3), (4-chloro-2-methylphenoxy)acetic acid 2-ethylhexyl ester (CAS NO: 29450-45-1), 2,4-dichlorophenoxyacetic acid butyl ester (CAS NO: 94-80-4), 2,4-dichlorophenoxyacetic acid

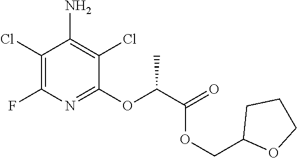 [SMILES: C[C@@H](Oc1nc(F)c(Cl)c(N)c1Cl)C(=O)OCC1CCCO1]

isooctyl ester (CAS NO: 25168-26-7), (CAS NO: 2445983-82-2), (2,4-dichlorophenoxy)acetic acid (CAS NO: 94-75-7), (2-methyl-4-chlorophenoxy)acetic acid dimethylamine salt (CAS NO: 2039-46-5), triaziflam (CAS NO: 131475-57-5), indaziflam (CAS NO: 950782-86-2), aclonifen (CAS NO: 74070-46-5), cinmethylin (CAS NO: 87818-31-3), tetflupyrolimet (CAS NO: 2053901-33-8), and oxaziclomefone (CAS NO: 153197-14-9). Wherein, the active ingredient A to the active ingredient B in the herbicidal composition is in a weight ratio of 1:1000-1000:1, 1:800-800:1, 1:600-600:1, 1:500-500:1, 1:400˜400:1, 1:300˜300:1, 1:200˜200:1, 1:180˜180:1, 1:150˜150:1, 1:120˜120:1, 1:100˜100:1, 1:80˜80:1, 1:50˜50:1, 1:30˜30:1, 1:20˜20:1, 1:10˜10:1, 1:5˜1:1 or 1:1˜5:1. The ingredients A and B together account for 1-95% of the total weight of the herbicidal composition, preferably 10-80%.

Generally, if the herbicidal compounds as described herein, which can be employed in the context of the present invention are capable of forming geometrical isomers, for example, E/Z isomers, it is possible to use both, the pure isomers and mixtures thereof, in the compositions according to the invention. If the herbicidal compounds as described herein have one or more centers of chirality and, as a consequence, are present as enantiomers or diastereomers, it is possible to use both, the pure enantiomers and diastereomers and their mixtures, in the compositions according to the invention. If the herbicidal compounds as described herein have ionizable functional groups, they can also be employed in the form of their agriculturally acceptable salts. In general, the salts of those cations and the acid addition salts of those acids are suitable, whose cations and anions, respectively, have no adverse effect on the activity of the active compounds. Preferred cations are the ions of alkali metals, preferably of lithium, sodium and potassium; ions of alkaline earth metals, preferably of calcium and magnesium; and ions of transition metals, preferably of manganese, copper, zinc and iron; further ammonium and substituted ammonium in which one to four hydrogen atoms are replaced by C₁-C₄-alkyl, hydroxy-C₁-C₄-alkyl, C₁-C₄-alkoxy-C₁-C₄-alkyl, hydroxy-C₁-C₄-alkoxy-C₁-C₄-alkyl, phenyl or benzyl, preferably ammonium, methylammonium, isopropylammonium, dimethylammonium, diisopropylammonium, trimethylammonium, heptylammonium, dodecylammonium, tetradecylammonium, tetramethylammonium, tetraethylammonium, tetrabutylammonium, 2-hydroxyethylammonium (olamine salt), 2-(2-hydroxyeth-1-oxy)eth-1-ylammonium (diglycolamine salt), di(2-hydroxyeth-1-yl)ammonium (diolamine salt), tris(2-hydroxyethyl)ammonium (trolamine salt), tris(2-hydroxypropyl)ammonium, benzyltrimethylammonium, benzyltriethylammonium, N,N,N-trimethylethanolammonium (choline salt), furthermore, phosphonium ions, sulfonium ions, preferably tri(C₁-C₄-alkyl)sulfonium such as trimethylsulfonium, and sulfoxonium ions, preferably tri(C₁-C₄-alkyl)sulfoxonium, and finally polyamines such as N,N-bis-(3-aminopropyl)methylamine and diethylenetriamine. The available anions of acid addition salts are primarily chloride, bromide, fluoride, iodide, hydrogensulfate, methylsulfate, sulfate, dihydrogenphosphate, hydrogenphosphate, nitrate, bicarbonate, carbonate, hexafluorosilicate, hexafluorophosphate, benzoate and also the anions of C₁-C₄-alkanoic acids, preferably formate, acetate, propionate and butyrate.

The herbicidal compounds as described herein having a carboxyl group can be employed in the form of the acid, in the form of an agriculturally suitable salt as mentioned above or else in the form of an agriculturally acceptable derivative, for example as amides such as mono- and di-C₁-C₆-alkylamides or arylamides, as esters, for example allyl esters, propargyl esters, C₁-C₁₀-alkyl esters, alkoxyalkyl esters, tefuryl ((tetrahydrofuran-2-yl)methyl) esters and also as thioesters, for example C₁-C₁₀-alkylthio esters. Preferred mono- and di-C₁-C₆-alkylamides are the methyl and the dimethylamides. Preferred arylamides are, for example, the anilides and the 2-chloroanilides. Preferred alkyl esters are, for example, methyl, ethyl, propyl, isopropyl, butyl, isobutyl, pentyl, mexyl (1-methylhexyl), meptyl (1-methylheptyl), heptyl, octyl or isooctyl (2-ethylhexyl) esters. Preferred C₁-C₄-alkoxy-C₁-C₄-alkyl esters are linear or branched C₁-C₄-alkoxy ethyl esters, for example 2-methoxyethyl, 2-ethoxyethyl, 2-butoxyethyl (butotyl), 2-butoxypropyl or 3-butoxypropyl ester. An example of a linear or branched C₁-C₁₀-alkylthio ester is ethyl thioester.

For use, the commercially available formulations are, if needed, diluted in customary manners, for example using water in the case of wettable powders, emulsifiable concentrates, dispersions and water-dispersible granules. Products in the form of dusts, granules for soil application, or solutions for broadcasting and spray are usually not further diluted with other inert substances prior to use. The application amount of the compounds of the formula I required varies with the external conditions such as temperature, humidity, the nature of the herbicide used and the like. It can vary within wide limits, for example between 0.001 and 1.0 kg a.i./ha or more of active substances, but it is preferably between 0.005 and 750 g a.i./ha.

### SPECIFIC MODE FOR CARRYING OUT THE INVENTION

The following embodiments are used to illustrate the present invention in detail and should not be taken as any limit to the present invention. The scope of the invention would be explained through the Claims.

In view of economy and variety of the compounds, we preferably synthesized several compounds, part of which are listed in the following Table 1. The specific structure and information of a certain compound are shown in Table 1. The compounds in Table 1 are listed for further explications of the present invention, rather than any limit to the present invention. The subject of the present invention should not be interpreted by those skilled in the art as being limited to the following compounds.

TABLE 1
Structures and ¹H NMR data of compounds

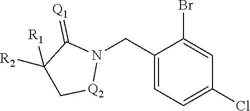

| NO. | R₁ | R₂ | Q₁ | Q₂ | ¹H NMR |
|---|---|---|---|---|---|
| 1 | Me | Me | O | O | ¹H NMR (500 MHZ, DMSO-d₆) |
| | | | | | δ 7.81 (d, J = 2.0 Hz, 1H), 7.48- |
| | | | | | 7.55 (m, 1H), 7.41 (d, J = 8.5 Hz, |
| | | | | | 1H), 4.72 (s, 2H), 4.07 (s, 2H), |
| | | | | | 1.16 (s, 6H). |
| 2 | Me | Me | S | O | |
| 3 | Me | Me | O | S | |
| 4 | Me | Me | S | S | |
| 5 | Et | Et | O | O | |

Several methods for preparing the compounds of the invention will be explained in detail in the following schemes and embodiments. The material is commercially available or can be prepared through known method reported in literatures or shown in the detailed explanation. Those skilled in the art should understand that the compounds of the invention can also be synthesized by other synthetic routes. Although the specific materials and reaction conditions in the synthetic route have been explicated in the following text, they are still easy to be replaced by other similar materials and conditions. Various isomers and the like of the compounds that were produced by the modifications or variations of the preparation method of the present invention are all included in the scope of the present invention. In addition, the following preparation method can be further modified according to the disclosures of the present invention by using common chemical method known to those skilled in the art, for example, protection of suitable groups in the process of the reaction, etc.

The following examples of methods can be used to improve further understanding of the preparation method of the present invention. The specific materials, classification and conditions have been determined to be further explications of the present invention, not to be any limit of the reasonable scope thereof. Reagents of the synthetic compounds showed in the following table can either be purchased from the market or easily prepared by those skilled in the art.

Examples of representative compounds are as follows. The synthesis methods of other compounds are similar and will not be described in detail here.

### 1. Synthesis of Compound 1

Compound 1-1 (284 mg, 1.0 mmol, 1.0 eq.) was dissolved in 10 mL of acetonitrile at 25° C., then added with Compound 1-2 (115 mg, 1.0 mmol, 1.0 eq.) and K₂CO₃ (207 mg, 1.5 mmol, 1.5 eq.), and the reaction solution was heated to 50° C. and reacted at this temperature for 1 h. After the reaction was completed, the reaction system was quenched by adding water, extracted with ethyl acetate, dried over anhydrous sodium sulfate, filtered, concentrated, and separated by column chromatography to obtain the target product 1 (232.5 mg, white solid, 73% yield).

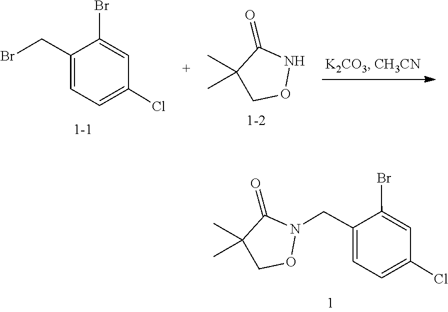

### Biological Activity Evaluation:

### (1) Experiment on Weeding Effect in Post-Emergence Stage:

The activity level criteria for plant damage (i.e., growth control rate) are as follows:

Level 5: growth control rate is greater than or equal to 85%;

Level 4: growth control rate is greater than or equal to 60% and less than 85%;

Level 3: growth control rate is greater than or equal to 40% and less than 60%;

Level 2: growth control rate is greater than or equal to 20% and less than 40%;

Level 1: growth control rate is greater than or equal to 5% and less than 20%;

Level 0: growth control rate is less than 5%.

The above growth control rates are fresh weight control rates.

Monocotyledonous and dicotyledonous weed seeds (Descurainia sophia, Capsella bursa-pastoris, Abutilon theophrasti, Galium aparine, Stellaria media, Lithospermum arvense, Rorippa indica, Alopecurus aequalis, Alopecurus japonicus, Eleusine indica, Beckmannia syzigachne, Sclerochloa dura, Phleum paniculatum, Veronica didyma, Bromus japonicus, Aegilops tauschii, Phalaris arundinacea, Amaranthus retroflexus, Chenopodiaceae, Commelina communis, Sonchus arvensis, Convolvulus arvensis, Cirsium setosum, Solanumnigrum, Acalypha australis, Digitaria sanguinalis, Echinochloa crusgalli, Setaria viridis, Setaria glauca, Leptochloa chinensis, Monochoria vaginalis, Sagittaria trifolia, Scirpus juncoides, Cyperus rotundus, Cyperus iria, Cyperus difformis, Fimbristylis, Portulaca oleracea, Xanthium sibiricum, Pharbitis nil, Conyza Canadensis, etc.) and major crop seeds (wheat, corn, rice, soybean, cotton, oilseed rape, millet, sorghum, potato, sesame, ricinus, etc.) were placed in plastic pots filled with soil, then covered with 0.5-2 cm of soil, allowed to grow in a good greenhouse environment. Two weeks after sowing, the test plants were treated in the 2-3 leaf stage. The tested compounds of the present invention were respectively dissolved in acetone, then added with Tween 80 and 1.5 liter/ha of emulsifiable concentrate of methyl oleate as synergist, diluted with a certain amount of water to obtain a solution of a certain concentration, and sprayed by a spray tower onto the plants. After the application, the plants were cultured for 3 weeks in the greenhouse, and then the experimental results of the weeding were recorded. The doses of the used compounds were 45, 90, 180, 360, 720 g a.i./ha, and the averages were obtained from three replicates. Representative data are listed in Table 2.

TABLE 2
Results on weeding effect in post-emergence stage (720 g a.i./ha)

| Compound | Digitaria | Leptochloa | Abutilon | Alopecurus | | |
|---|---|---|---|---|---|---|
| NO. | sanguinalis | chinensis | theophrasti | japonicus | rice | wheat |
| 1 | 5 | 5 | 5 | 4 | 1 | 1 |
| Control | 3 | 2 | 4 | 3 | 2 | 3 |
| compound A | | | | | | |
| Control | 3 | 4 | 3 | 4 | 3 | 4 |
| compound B | | | | | | |

Note: Control compound A:

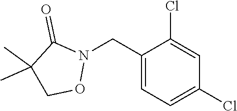 [SMILES: CC1(C)CON(Cc2ccc(Cl)cc2Cl)C1=O]

(bixlozone); Control compound B: clomazone.

The above experimental results indicate that the present compound, compared with commercialized clomazone and bixlozone, has better crop safety, especially in wheat and rice, and also has comparable or even better herbicidal activity against key weeds such as crabgrass (Digitaria sanguinalis), sprangletop (Leptochloa chinensis), velvetleaf (Abutilon theophrasti), Japanese foxtail (Alopecurus japonicus), thus enabling a better selectivity in crops.

### (2) Experiment on Weeding Effect in Pre-Emergence Stage:

The activity level criteria for plant damage (i.e., growth control rate) are as follows:

Level 9: completely dead;

Level 8: growth control rate is greater than or equal to 80% and less than 100%;

Level 7: growth control rate is greater than or equal to 70% and less than 80%;

Level 6: growth control rate is greater than or equal to 60% and less than 70%;

Level 5: growth control rate is greater than or equal to 50% and less than 60%;

Level 4: growth control rate is greater than or equal to 40% and less than 50%;

Level 3: growth control rate is greater than or equal to 30% and less than 40%;

Level 2: growth control rate is greater than or equal to 15% and less than 30%;

Level 1: growth control rate is greater than 0% and less than 15%;

Level 0: no effect.

The above growth control rates are fresh weight control rates.

Monocotyledonous and dicotyledonous weed seeds (Descurainia sophia, Capsella bursa-pastoris, Abutilon theophrasti, Galium aparine, Stellaria media, Lithospermum arvense, Rorippa indica, Alopecurus aequalis, Alopecurus japonicus, Eleusine indica, Beckmannia syzigachne, Sclerochloa dura, Phleum paniculatum, Veronica didyma, Bromus japonicus, Aegilops tauschii, Phalaris arundinacea, Amaranthus retroflexus, Chenopodiaceae, Commelina communis, Sonchus arvensis, Convolvulus arvensis, Cirsium setosum, Solanumnigrum, Acalypha australis, Digitaria sanguinalis, Echinochloa crusgalli, Setaria viridis, Setaria glauca, Leptochloa chinensis, Monochoria vaginalis, Sagittaria trifolia, Scirpus juncoides, Cyperus rotundus, Cyperus iria, Cyperus difformis, Fimbristylis, Portulaca oleracea, Xanthium sibiricum, Pharbitis nil, Conyza Canadensis, etc.) and major crop seeds (wheat, corn, rice, soybean, cotton, oilseed rape, millet, sorghum, potato, sesame, ricinus, etc.) were placed in plastic pots filled with soil, then covered with 0.5-2 cm of soil, allowed to grow in a good greenhouse environment. The test plants were treated on the day of sowing. The tested compounds of the present invention were respectively dissolved in acetone, then added with Tween 80, diluted with a certain amount of water to obtain a solution of a certain concentration, and sprayed by a spray tower onto the plants. After the application, the plants were cultured for 2 weeks in the greenhouse, and then the experimental results of the weeding were recorded. The doses of the used compounds were 3, 6, 12, 24, 48 g ai./mu (1 mu=1/15 ha), and the averages were obtained from three replicates. Representative data are listed in Table 3.

TABLE 3
Results on weeding effect in pre-emergence stage

| | | | | Control | Control |
|---|---|---|---|---|---|
| Compound NO. | | | 1 | compound A | compound B |
| corn | 24 | g a.i./mu | 0 | 2 | 3 |
| soy bean | 24 | g a.i./mu | 0 | 1 | 2 |
| | 48 | g a.i./mu | 0 | 2 | 3 |
| peanut | 48 | g a.i./mu | 0 | 1 | 2 |
| cotton | 12 | g a.i./mu | 0 | 1 | 2 |
| | 24 | g a.i./mu | 0 | 2 | 2 |
| rice | 6 | g a.i./mu | 0 | 1 | 2 |
| | 12 | g a.i./mu | 0 | 2 | 2 |
| | 24 | g a.i./mu | 1 | 4 | 5 |
| wheat | 6 | g a.i./mu | 0 | 1 | 3 |
| | 12 | g a.i./mu | 0 | 1 | 4 |
| | 24 | g a.i./mu | 0 | 2 | 6 |
| Echinochloa | 3 | g a.i./mu | 8 | 7 | 8 |
| crusgalli | 6 | g a.i./mu | 9 | 9 | 9 |
| | 12 | g a.i./mu | 9 | 9 | 9 |
| | 24 | g a.i./mu | 9 | 9 | 9 |
| Leptochloa | 3 | g a.i./mu | 9 | 6 | 0 |
| chinensis | 6 | g a.i./mu | 9 | 9 | 4 |
| | 12 | g a.i./mu | 9 | 9 | 9 |
| | 24 | g a.i./mu | 9 | 9 | 9 |
| Lolium | 6 | g a.i./mu | 2 | 2 | 1 |
| perenne | 24 | g a.i./mu | 9 | 9 | 9 |
| Veronica | 6 | g a.i./mu | 8 | 8 | 8 |
| didyma | 12 | g a.i./mu | 9 | 9 | 9 |
| | 24 | g a.i./mu | 9 | 9 | 9 |
| Descurainia | 3 | g a.i./mu | 7 | 4 | 2 |
| sophia | 6 | g a.i./mu | 9 | 4 | 3 |
| | 12 | g a.i./mu | 9 | 9 | 7 |
| | 24 | g a.i./mu | 9 | 9 | 7 |

It can be concluded that the present compound, compared with commercialized clomazone and bixlozone, has greater commercial value because of its superior safety in soil treatment and comparable or even better herbicidal activity, especially against key weeds in wheat including perennial ryegrass (Lolium perenne), speedwell (Veronica didyma) and flixweed (Descurainia sophia), as well as key weeds in rice including barnyard grass (Echinochloa crusgalli) and sprangletop (Leptochloa chinensis).

### (3) Volatility Evaluation

Since the commercialization of clomazone, there have been a large number of commercial complaints and compensations due to pesticide drift caused by its volatilization. It has been reported that 48% of clomazone emulsifiable concentrate applied in windy conditions may result in bleaching injury to poplars and willows within 1.6 kilometers downwind, and tend to bring unacceptable risks to a variety of vegetables, fruit trees, wolfberry, etc., which seriously restricts its commercial value; however, the present compounds have better anti-volatility and obviously higher safety.

Stellaria media is very sensitive to clomazone compounds and is a good indicator for bleaching compound volatility. Untreated control plates, clomazone emulsifiable concentrate and bixlozone emulsifiable concentrate formulations were included in each test. These formulations are the reference standards against which the experimental formulations are compared. Four-inch plastic pots were fitted with basket style coffee filters which were cut to size and placed at the bottom of each pot to cover the drainage holes and to stop soil from exiting from the bottom of the pot. Each pot was filled with sandy loam soil that was sieved using a #10 mesh sieve to remove any large soil particles and debris. Filled soil pots were lightly watered prior to treatment application. The test solutions were applied to the soil surface using a sprayer, two replicates per test solution, at a dosage of 0.25 kg ai/ha.

Each replicate test was set up by placing an empty 4-inch plastic pot on top of a greenhouse. Pots containing mature Stellaria media, about 2 inches in height, were placed around the empty pot in an 8-spoke pattern. Four pots of the mature Stellaria media were placed at the 3, 6, 9 and 12 o'clock positions of the spoke, while the other four pots were placed at the 1:30, 4:30, 7:30 and 10:30 o'clock positions of the spoke. Once all of the pots were in place, the empty 4-inch pot from each set-up was removed and a treated 4-inch soil pot was put in its place. The untreated Stellaria media plants were evaluated for phytotoxic injury 14 days after treatment. Volatility was evaluated by measuring the distance from the center of the treated soil pot to the distal point where bleaching effects were observed on each spoke. All eight spokes for each treatment were evaluated. The amount of volatilization was determined by calculating the total square centimeters of phytotoxic injury per treatment. Clomazone emulsifiable concentrate was considered to be volatility control as this formulation consists of 100% free clomazone. The volatility reported is a relative value to the volatility of clomazone emulsifiable concentrate which was normalized to 100%. Table 4 below summarizes the average of the volatility evaluations.

TABLE 4
Volatility Evaluations

| | Dosage of | % Volatility relative to |
|---|---|---|
| | application g | clomazone emulsifiable |
| Test formulation | a.i./ha | concentrate |
| Control | 0 | 0 |
| clomazone emulsifiable concentrate | 250 | 100 |
| (40% clomazone + 5% calcium dodecylbenzene sulfonate + | | |
| 9% phenethyl phenol polyoxyethylene ether + 150# | | |
| solvent oil as balance.) | | |
| bixlozone emulsifiable concentrate (40% bixlozone + 5% | 250 | 35 |
| calcium dodecylbenzene sulfonate + 9% phenethyl phenol | | |
| polyoxyethylene ether + 150# solvent oil as balance.) | | |
| Compound 1 emulsifiable concentrate (40% Compound 1 + | 250 | 15 |
| 5% calcium dodecylbenzene sulfonate + 9% phenethyl | | |
| phenol polyoxyethylene ether + 150# solvent oil as | | |
| balance.) | | |

The volatility of the compound of the present invention is significantly reduced compared to clomazone and bixlozone emulsifiable concentrates.

### (4) Transplanted Rice Safety Evaluation and Weed Control Effect Evaluation in Rice Field:

Rice field soil was loaded into a 1/1,000,000 ha pot. The seeds of Echinochloa crusgalli, Scirpus juncoides, and Bidens tripartita L. were sowed and gently covered with soil, then placed in a greenhouse with water storage of 0.5-1 cm deep. The tuber of Sagittaria trifolia was planted in the next day or 2 days later. The water storage was kept at a depth of 3-4 cm thereafter. The weeds were treated by homogeneously dripping the WP or SC water diluents of the compounds of the present invention prepared according to the common preparation methods with a pipette to the specified effective amount when the Echinochloa crusgalli, Scirpus juncoides, and Bidens tripartita L. reached their 0.5 leaf stages and the Sagittaria trifolia reached the time point of the primary leaf stage.

In addition, the rice field soil that loaded into the 1/1,000,000 ha pot was leveled to keep water storage at a depth of 3-4 cm. The rice (japonica rice) at 3-leaf stage was transplanted at 3-cm transplanting depth the next day. The compound of the present invention was treated by the same way 5 days after the transplantation.

The development condition of Echinochloa crusgalli, Scirpus juncoides, Bidens tripartita L., and Sagittaria trifolia 14 days after the treatment of the compound of the invention and the development condition of rice 21 days after the treatment of the compound of the invention were respectively observed with the naked eye. The weed control effect was evaluated according to the above activity level criteria. The Compound 1 showed excellent activity and selectivity.

Note: The seeds of Echinochloa crusgalli, Scirpus juncoides and Bidens tripartita L., and Sagittaria trifolia were collected from Heilongjiang Province of China. The tests indicated that the weeds were resistant to pyrazosulfuron-ethyl of regular doses.

### (5) Field Test:

Trial result shows that Compound 1, applied after planting and pre-emergence at the dosage of 300 g a.i./ha, is safe to a variety of crops such as spinach, radish, Chinese cabbage, sesame, rape, cucumber, watermelon, melon, pumpkin, cassava, soybean, kidney bean, cowpea, common bean, hyacinth bean, sponge gourd, sorghum, garlic, rice, corn, cauliflower, cabbage and coriander. Meanwhile it effectively controls major weeds such as barnyard grass (Echinochloa crusgalli), sprangletop (Leptochloa chinensis), crabgrass (Digitaria sanguinalis), flixweed (Descurainia Sophia), shepherd's-purse (Capsella bursa-pastoris), cleavers (Galium aparine), chickweed (Stellaria media), perennial ryegrass (Lolium perenne), bromegrass (Bromus japonicas), black-grass (Alopecurus myosuroides), etc.

### (6) Activity Test of the Compositions:

The desired active ingredient B was purchased from a reagent company or synthesized by a known method. The technical materials were all dissolved in acetone and diluted with an aqueous solution containing 0.10% emulsifier Tween-80 which were to be used immediately after dilution.

### (A) Post-Emergence Treatment by Performing Foliage Spray:

Weeds were cultivated by a pot culture method. A 180×140 mm plastic nutritional bowl containing topsoil (taking up 4/5 of the bowl) collected from the field was placed in an enamel pan, wherein the soil had been air-dried and sieved and had an initial moisture content of 20%. Weed seeds with plump and uniform grains were selected, soaked in lukewarm water at 25° C. for 6 hours, and germinated in a 28° C. biochemical incubator (in darkness). The weed seeds that had just germinated were evenly placed on the surface of the soil and then covered with 0.5-1 cm soil according to the sizes of seeds.

The culture was carried out in a controllable sunlight greenhouse at 20 to 30° C., in natural light, and with relative humidity of 57% to 72%. The soil was loam with an organic matter content of 1.63%, a pH value of 7.1, alkali-hydrolyzable nitrogen of 84.3 mg/kg, rapidly available phosphorus of 38.5 mg/kg, and rapidly available potassium of 82.1 mg/kg.

Each treatment was repeated 4 times. Four pots with 20 weed seeds per pot were treated each time.

The agents were used for only once in total in the experiment. In the stage of 1.5-2 leaves, the weeds were thinned out to maintain 10 weeds per pot. Ten weeds were kept in each treatment, then continued to be cultured until Echinochloa crus-galli var. zelayensis reached the stage of 2-3 leaves, Cirsium setosum reached the stage of 5-6 leaves, Galium aparine reached the stage of 3-4 whorl leaves, Amaranthus retroflexus reached the stage of 3-5 leaves, Echinochloa crusgalli, Lolium multiflorum and Digitaria sanguinalis reached the stage of 3 leaves with one leaf not fully expanding for further treatment.

The well-cultured test materials were evenly placed on a platform with an area of 0.5 m², and solution of agents was sprayed on the stems and leaves thereof by the 3WP-2000-type walking spray tower at a dosage of 450 kg/ha and at a spray pressure of 0.3 MPa. After all the solution was sprayed, the valve was closed. After 30 seconds, the door of the spray tower was opened, and the nutritional bowl was taken out. Then the valve was opened, and the spray tube was cleaned by spraying 50 ml of water. After the treatment, the weeds were routinely cultured in a greenhouse.

### (B) Soil Sealing Treatment:

Weeds are cultivated in a controllable sunlight greenhouse at 20 to 30° C., in natural light, and with relative humidity of 57% to 72%. The soil was loam with an organic matter content of 1.63%, a pH value of 7.1, alkali-hydrolyzable nitrogen of 84.3 mg/kg, rapidly available phosphorus of 38.5 mg/kg, and rapidly available potassium of 82.1 mg/kg. The test soil was filled quantitatively to ¾ of the pots and then watered from the bottom of the pots to completely wet the soil to saturation. The test weed seeds were accelerated to just germinate, and then evenly and quantitatively sowed on the surface, then covered with 0.5-1 cm soil according to the seed size, and ready for use 72 hours after sowing.

Each treatment was repeated 4 times. Four pots with 20 weed seeds per pot were treated each time.

The well-sowed test materials were evenly placed on a platform with an area of 0.5 m², and solution of agents was sprayed on the soil by the 3WP-2000-type walking spray tower at a dosage of 450 kg/ha and at a spray pressure of 0.3 MPa. After all the solution was sprayed, the valve was closed. After 30 seconds, the door of the spray tower was opened, and the nutritional bowl was taken out. Then the valve was opened, and the spray tube was cleaned by spraying 50 ml of water.

### (C) Data Investigation and Statistical Analysis:

A method for investigating absolute numbers was employed. Whole seedlings of survival weeds were cut off with a blade along the soil surface, and the fresh weight of the weeds was weighed with an analytical balance. For dead weeds, the fresh weight thereof was zero.

The investigation was performed 21 days after the treatment for only once in total.

The theoretical fresh weight inhibition rate of a combination of two active ingredients in each group was calculated by the Gowing method (E0=X+Y−X*Y/100), and then compared with an actually measured inhibition rate (E), thereby the effect of the combination (hereafter referred to as combined effect) on weeds was evaluated: the value of E−E0, which was greater than 10%, corresponded to a synergistic effect; the value of E−E0, which was less than −10%, corresponded to an antagonistic effect; and the value of E-E0, which was from −10% to Ra, corresponded to an additional effect. An optimum ratio of the two active ingredients was determined by the actual control effect, characteristics of herbicides, and balance of a corresponding formula. Wherein, in the formula, X represented the fresh weight inhibition rate of the active ingredient A in a dosage of P, and Y represented the fresh weight inhibition rate of the active ingredient B in a dosage of Q. The statistical results were shown in the Table 5.

TABLE 5
Actual control effect and combined effect of a combination of Compound 1
on weeds

| | | | | | Control | Control | Actual | | |
|---|---|---|---|---|---|---|---|---|---|
| | | | | | effect | effect | control | | |
| | | | | | (%) of | (%) of | effect | Theoretical | |
| | | | | | A | B | of A + B | control | E |
| | | Foliage/ | | | applied | applied | (%) | effect of | (A + B)- |
| Components | | Soil | Dose | | alone | alone | E | A + B (%) | E0 |
| A + B | Weed | F/S | g a.i./mu | Ratio | (A) | (B) | (A + B) | E0 (A + B) | (A + B) |
| 1 + acetochlor | Lolium | S | 8 + 12 | 2:3 | 53.2 | 36.5 | 91.4 | 70.3 | 21.1 |
| | multiflorum | | 8 + 24 | 1:3 | 53.2 | 45.7 | 98.7 | 74.6 | 24.1 |
| 1 + pretilachlor | Lolium | S | 8 + 8 | 1:1 | 53.2 | 23.4 | 89.2 | 64.2 | 25.0 |
| | multiflorum | | 8 + 16 | 1:2 | 53.2 | 50.9 | 92.1 | 77.0 | 15.1 |
| 1 + butachlor | Lolium | S | 8 + 16 | 1:2 | 53.2 | 28.8 | 86.6 | 66.7 | 19.9 |
| | multiflorum | | 8 + 32 | 1:4 | 53.2 | 46.4 | 93.2 | 74.9 | 18.3 |
| 1 + S- | Lolium | S | 8 + 12 | 2:3 | 53.2 | 32.1 | 81.5 | 68.2 | 13.3 |
| metolachlor | multiflorum | | 8 + 24 | 1:3 | 53.2 | 53.6 | 93.1 | 78.3 | 14.8 |
| 1 + mefenacet | Lolium | S | 8 + 16 | 1:2 | 53.2 | 18.5 | 94.4 | 61.9 | 32.5 |
| | multiflorum | | 8 + 32 | 1:4 | 53.2 | 38.3 | 83.2 | 71.1 | 12.1 |
| 1 + pyroxasulfone | Lolium | S | 8 + 2 | 4:1 | 53.2 | 24.7 | 89.6 | 64.8 | 24.8 |
| | multiflorum | | 8 + 4 | 2:1 | 53.2 | 49.2 | 94.3 | 76.2 | 18.1 |
| 1 + atrazine | Lolium | S | 8 + 20 | 2:5 | 53.2 | 18.9 | 79.4 | 62.0 | 17.4 |
| | multiflorum | | 8 + 40 | 1:5 | 53.2 | 36.6 | 85.8 | 70.3 | 15.5 |
| 1 + ametryne | Lolium | S | 8 + 20 | 2:5 | 53.2 | 18.1 | 84.7 | 61.7 | 23.0 |
| | multiflorum | | 8 + 40 | 1:5 | 53.2 | 31.6 | 90.5 | 68.0 | 22.5 |
| 1 + diuron | Lolium | S | 8 + 40 | 1:5 | 53.2 | 39.4 | 87.4 | 71.6 | 15.8 |
| | multiflorum | | 8 + 80 | 1:10 | 53.2 | 65.3 | 99.2 | 83.8 | 15.4 |
| 1 + pendimethalin | Lolium | S | 8 + 16 | 1:2 | 53.2 | 23.6 | 78.5 | 64.2 | 14.3 |
| | multiflorum | | 8 + 32 | 1:4 | 53.2 | 42.7 | 89.4 | 73.2 | 16.2 |
| 1 + butralin | Lolium | S | 8 + 16 | 1:2 | 53.2 | 19.5 | 80.1 | 62.3 | 17.8 |
| | multiflorum | | 8 + 32 | 1:4 | 53.2 | 35.8 | 95.9 | 70.0 | 25.9 |
| 1 + propanil | Echinochloa | F | 16 + 16 | 1:1 | 43.6 | 16.8 | 74.6 | 53.1 | 21.5 |
| | crusgalli | | 16 + 32 | 1:2 | 43.6 | 31.2 | 86.9 | 61.2 | 25.7 |
| | var. | | | | | | | | |
| | zelayensis | | | | | | | | |
| 1 + quinclorac | Echinochloa | F | 16 + 4 | 4:1 | 43.6 | 22.5 | 81.2 | 56.3 | 24.9 |
| | crusgalli | | 16 + 8 | 2:1 | 43.6 | 41.4 | 91.7 | 66.9 | 24.8 |
| | var. | | | | | | | | |
| | zelayensis | | | | | | | | |
| 1 + bentazon | Cirsium | F | 32 + 8 | 4:1 | 64.2 | 12.4 | 87.6 | 68.6 | 19.0 |
| | setosum | | 32 + 16 | 2:1 | 64.2 | 25.8 | 93.8 | 73.4 | 20.4 |
| 1 + napropamide | Echinochloa | S | 8 + 8 | 1:1 | 50.7 | 30.1 | 79.4 | 65.5 | 13.9 |
| | crusgalli | | 8 + 16 | 1:2 | 50.7 | 42.3 | 86.2 | 71.6 | 14.6 |
| 1 + ipfencarbazone | Echinochloa | S | 8 + 2 | 4:1 | 50.7 | 41.5 | 83.1 | 71.2 | 11.9 |
| | crusgalli | | 8 + 4 | 2:1 | 50.7 | 57.6 | 91.4 | 79.1 | 12.3 |
| 1 + fentrazamide | Echinochloa | S | 8 + 16 | 1:2 | 50.7 | 32.5 | 79.9 | 66.7 | 13.2 |
| | crusgalli | | 8 + 32 | 1:4 | 50.7 | 50.8 | 87.2 | 75.7 | 11.5 |
| 1 + bromobutide | Echinochloa | S | 8 + 16 | 1:2 | 50.7 | 37.3 | 83.5 | 69.1 | 14.4 |
| | crusgalli | | 8 + 32 | 1:4 | 50.7 | 53.6 | 90.6 | 77.1 | 13.5 |
| 1 + molinate | Echinochloa | S | 8 + 20 | 2:5 | 50.7 | 39.6 | 85.7 | 70.2 | 15.5 |
| | crusgalli | | 8 + 40 | 1:5 | 50.7 | 51.7 | 91.1 | 76.2 | 14.9 |
| 1 + flufenacet | Echinochloa | S | 8 + 6 | 4:3 | 50.7 | 29.8 | 80.1 | 65.4 | 14.7 |
| | crusgalli | | | | | | | | |
| | | | 8 + 12 | 2:3 | 50.7 | 37.5 | 82.4 | 69.2 | 13.2 |
| 1 + anilofos | Echinochloa | S | 8 + 2 | 4:1 | 50.7 | 23.1 | 75.6 | 62.1 | 13.5 |
| | crusgalli | | | | | | | | |
| | | | 8 + 4 | 2:1 | 50.7 | 32.6 | 80.4 | 66.8 | 13.6 |
| 1 + prosulfocarb | Galium | F | 16 + 50 | 8:25 | 55.4 | 40.5 | 85.7 | 73.5 | 12.2 |
| | aparine | | 16 + 100 | 4:25 | 55.4 | 56.1 | 92.2 | 80.4 | 11.8 |
| 1 + dimethenamid | Echinochloa | S | 8 + 6 | 4:3 | 50.7 | 31.5 | 79.3 | 66.2 | 13.1 |
| | crusgalli | | 8 + 12 | 2:3 | 50.7 | 43.7 | 86.8 | 72.2 | 14.6 |
| 1 + | Echinochloa | S | 8 + 4 | 2:1 | 50.7 | 34.2 | 80.1 | 67.6 | 12.5 |
| dimethenamid- | crusgalli | | 8 + 8 | 1:1 | 50.7 | 47.5 | 87.9 | 74.1 | 13.8 |
| P | | | | | | | | | |
| 1 + isoproturon | Alopecurus | S | 8 + 8 | 1:1 | 39.4 | 41.5 | 76.2 | 64.5 | 11.7 |
| | japonicus | | 8 + 16 | 1:2 | 39.4 | 53.6 | 85.4 | 71.9 | 13.5 |
| 1 + chlorotoluron | Alopecurus | S | 8 + 15 | 8:15 | 39.4 | 34.2 | 75.4 | 60.1 | 15.3 |
| | japonicus | | 8 + 30 | 4:15 | 39.4 | 58.3 | 89.1 | 74.7 | 14.4 |
| 1 + metribuzin | Amaranthus | S | 8 + 5 | 8:5 | 37.3 | 40.5 | 74.9 | 62.7 | 12.2 |
| | retroflexus | | 8 + 10 | 4:5 | 37.3 | 56.2 | 83.5 | 72.5 | 11.0 |
| 1 + terbutylazine | Echinochloa | S | 8 + 6 | 4:3 | 50.7 | 33.8 | 80.6 | 67.4 | 13.2 |
| | crusgalli | | 8 + 12 | 2:3 | 50.7 | 52.7 | 88.5 | 76.7 | 11.8 |
| 1 + simetryn | Potamogeton | S | 8 + 6 | 4:3 | 35.9 | 41.5 | 75.4 | 62.5 | 12.9 |
| | distinctus | | 8 + 12 | 2:3 | 35.9 | 58.3 | 87.8 | 73.3 | 14.5 |
| 1 + bromoxynil | Amaranthus | F | 8 + 6 | 4:3 | 36.7 | 38.2 | 73.3 | 60.9 | 12.4 |
| octanoate | retroflexus | | 8 + 12 | 2:3 | 36.7 | 49.5 | 81.6 | 68.0 | 13.6 |
| 1 + prometryne | Commelina | S | 8 + 6 | 4:3 | 44.1 | 30.5 | 77.3 | 61.1 | 16.2 |
| | communis | | 8 + 12 | 2:3 | 44.1 | 41.7 | 83.2 | 67.4 | 15.8 |
| 1 + amicarbazone | Echinochloa | F | 16 + 4 | 4:1 | 46.2 | 29.8 | 76.5 | 62.2 | 14.3 |
| | crusgalli | | 16 + 8 | 2:1 | 46.2 | 37.2 | 81.2 | 66.2 | 15.0 |
| 1 + terbutryne | Alopecurus | S | 8 + 12 | 2:3 | 39.4 | 32.4 | 72.8 | 59.0 | 13.8 |
| | japonicus | | 8 + 24 | 1:3 | 39.4 | 41.9 | 80.6 | 64.8 | 15.8 |
| 1 + diflufenican | Lolium | S | 8 + 1 | 8:1 | 53.2 | 37.1 | 83.3 | 70.6 | 12.7 |
| | multiflorum | | 8 + 2 | 4:1 | 53.2 | 52.4 | 92.1 | 77.7 | 14.4 |
| 1 + picolinafen | Galium | S | 8 + 0.5 | 16:1 | 37.2 | 40.8 | 78.6 | 62.8 | 15.8 |
| | aparine | | 8 + 1 | 8:1 | 37.2 | 55.7 | 85.3 | 72.2 | 13.1 |
| 1 + beflubutamid | Veronica | S | 8 + 1 | 8:1 | 48.9 | 42.6 | 85.2 | 70.7 | 14.5 |
| | didyma | | 8 + 2 | 4:1 | 48.9 | 57.9 | 92.7 | 78.5 | 14.2 |
| 1 + fluridone | Veronica | S | 8 + 1 | 8:1 | 48.9 | 37.2 | 83.5 | 67.9 | 15.6 |
| | didyma | | 8 + 2 | 4:1 | 48.9 | 50.4 | 90.7 | 74.7 | 16.0 |
| 1 + flurochloridone | Veronica | S | 8 + 4 | 2:1 | 48.9 | 45.1 | 85.9 | 71.9 | 14.0 |
| | didyma | | 8 + 8 | 1:1 | 48.9 | 60.8 | 93.5 | 80.0 | 13.5 |
| 1 + dithiopyr | Echinochloa | S | 8 + 4 | 2:1 | 50.7 | 40.1 | 83.4 | 70.5 | 12.9 |
| | crusgalli | | 8 + 8 | 1:1 | 50.7 | 53.2 | 90.5 | 76.9 | 13.6 |
| 1 + oxadiazon | Echinochloa | S | 8 + 4 | 2:1 | 50.7 | 37.2 | 82.4 | 69.0 | 13.4 |
| | crusgalli | | 8 + 8 | 1:1 | 50.7 | 51.9 | 88.5 | 76.3 | 12.2 |
| 1 + oxadiargy1 | Echinochloa | S | 8 + 0.5 | 16:1 | 50.7 | 36.2 | 83.1 | 68.5 | 14.6 |
| | crusgalli | | 8 + 1 | 8:1 | 50.7 | 47.9 | 89.7 | 74.3 | 15.4 |
| 1 + pentoxazone | Echinochloa | S | 8 + 2 | 4:1 | 50.7 | 40.1 | 83.9 | 70.5 | 13.4 |
| | crusgalli | | 8 + 4 | 2:1 | 50.7 | 57.3 | 90.4 | 78.9 | 11.5 |
| 1 + pyraclonil | Echinochloa | S | 8 + 1 | 8:1 | 50.7 | 22.5 | 77.1 | 61.8 | 15.3 |
| | crusgalli | | 8 + 2 | 4:1 | 50.7 | 43.6 | 86.4 | 72.2 | 14.2 |
| 1 + sulfentrazone | Echinochloa | S | 8 + 4 | 2:1 | 50.7 | 34.2 | 81.5 | 67.6 | 13.9 |
| | crusgalli | | 8 + 8 | 1:1 | 50.7 | 53.6 | 89.9 | 77.1 | 12.8 |
| 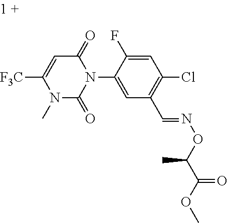 [SMILES: COC(=O)[C@@H](C)O/N=C/c1cc(-n2c(=O)cc(C(F)(F)F)n(C)c2=O)c(F)cc1Cl] | Echinochloa crusgalli | S | 8 + 0.5 8 + 1 | 16:1 8:1 | 50.7 50.7 | 31.5 42.3 | 82.4 89.7 | 66.2 71.6 | 16.2 18.1 |
| 1 + flumioxazin | Echinochloa | S | 8 + 0.5 | 16:1 | 50.7 | 32.8 | 80.6 | 66.9 | 13.7 |
| | crusgalli | | 8 + 1 | 8:1 | 50.7 | 45.1 | 87.8 | 72.9 | 14.9 |
| 1 + oxyfluorfen | Digitaria | S | 8 + 1 | 8:1 | 40.3 | 28.6 | 72.4 | 57.4 | 15.0 |
| | sanguinalis | | 8 + 2 | 4:1 | 40.3 | 39.5 | 80.7 | 63.9 | 16.8 |
| 1 + saflufenacil | Amaranthus | S | 8 + 0.25 | 32:1 | 37.3 | 35.2 | 73.5 | 59.4 | 14.1 |
| | retroflexus | | 8 + 0.5 | 16:1 | 37.3 | 43.8 | 78.2 | 64.8 | 13.4 |
| 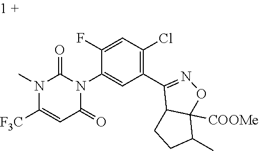 [SMILES: COC(=O)C12ON=C(c3cc(-n4c(=O)cc(C(F)(F)F)n(C)c4=O)c(F)cc3Cl)C1CCC2C] | Echinochloa crusgalli | S | 8 + 1 8 + 2 | 8:1 4:1 | 50.7 50.7 | 40.2 53.1 | 87.3 91.5 | 70.5 76.9 | 16.8 14.6 |
| 1 + tiafenacil | Echinochloa | F | 16 + 0.5 | 32:1 | 46.2 | 33.4 | 79.6 | 64.2 | 15.4 |
| | crusgalli | | 16 + 1 | 16:1 | 46.2 | 45.9 | 85.7 | 70.9 | 14.8 |
| 1 + trifludimoxazin | Lolium | S | 8 + 2 | 4:1 | 53.2 | 31.5 | 80.3 | 67.9 | 12.4 |
| | multiflorum | | 8 + 4 | 2:1 | 53.2 | 42.6 | 87.9 | 73.1 | 14.8 |
| 1 + fomesafen | Amaranthus | F | 16 + 4 | 4:1 | 36.7 | 38.1 | 75.4 | 60.8 | 14.6 |
| | retroflexus | | 16 + 8 | 2:1 | 36.7 | 47.5 | 81.9 | 66.8 | 15.1 |
| 1 + epyrifenacil | Amaranthus | S | 8 + 0.5 | 16:1 | 37.3 | 36.9 | 73.2 | 60.4 | 12.8 |
| | retroflexus | | 8 + 1 | 8:1 | 37.3 | 47.1 | 80.5 | 66.8 | 13.7 |
| 1 + benmicaozuo | Echinochloa | F | 16 + 1 | 16:1 | 46.2 | 37.1 | 81.6 | 66.2 | 15.4 |
| | crusgalli | | 16 + 2 | 8:1 | 46.2 | 45.3 | 85.4 | 70.6 | 14.8 |
| 1 + bensulfuron- | Cyperus | S | 8 + 0.5 | 16:1 | 34.5 | 42.6 | 81.7 | 62.4 | 19.3 |
| methyl | difformis | | 8 + 1 | 8:1 | 34.5 | 55.8 | 89.1 | 71.0 | 18.1 |
| 1 + ethoxysulfuron | Cyperus | S | 8 + 0.25 | 32:1 | 34.5 | 44.6 | 79.4 | 63.7 | 15.7 |
| | difformis | | 8 + 0.5 | 16:1 | 34.5 | 58.4 | 88.7 | 72.8 | 15.9 |
| 1 + pyrazosulfuron- | Cyperus | S | 8 + 0.25 | 32:1 | 34.5 | 44.8 | 83.7 | 63.8 | 19.9 |
| ethyl | difformis | | 8 + 0.5 | 16:1 | 34.5 | 59.7 | 92.4 | 73.6 | 18.8 |
| 1 + halosulfuron | Veronica | S | 8 + 0.5 | 16:1 | 48.9 | 34.8 | 84.7 | 66.7 | 18.0 |
| | didyma | | 8 + 1 | 8:1 | 48.9 | 54.6 | 90.6 | 76.8 | 13.8 |
| 1 + penoxsulam | Echinochloa | F | 16 + 0.5 | 32:1 | 46.2 | 52.7 | 88.6 | 74.6 | 14.0 |
| | crusgalli | | 16 + 1 | 16:1 | 46.2 | 61.7 | 92.7 | 79.4 | 13.3 |
| 1 + metazosulfuron | Echinochloa | S | 8 + 0.75 | 32:3 | 50.7 | 32.4 | 83.2 | 66.7 | 16.5 |
| | crusgalli | | 8 + 1.5 | 16:3 | 50.7 | 41.6 | 91.5 | 71.2 | 20.3 |
| 1 + propyrisulfuron | Echinochloa | S | 8 + 0.6 | 40:3 | 50.7 | 22.6 | 81.7 | 61.8 | 19.9 |
| | crusgalli | | 8 + 1.2 | 20:3 | 50.7 | 37.5 | 90.8 | 69.2 | 21.6 |
| 1 + triafamone | Echinochloa | S | 8 + 0.25 | 32:1 | 50.7 | 27.6 | 79.6 | 64.3 | 15.3 |
| | crusgalli | | 8 + 0.5 | 16:1 | 50.7 | 38.4 | 88.7 | 69.6 | 19.1 |
| 1 + bispyribac | Echinochloa | F | 16 + 0.6 | 80:3 | 46.2 | 30.7 | 84.6 | 62.7 | 21.9 |
| | crusgalli | | 16 + 1.2 | 40:3 | 46.2 | 42. | 90.5 | 69.2 | 21.3 |
| 1 + florasulam | Galium | F | 16 + 0.1 | 160:1 | 55.4 | 31.5 | 83.6 | 69.4 | 14.2 |
| | aparine | | 16 + 0.2 | 80:1 | 55.4 | 44.1 | 93.4 | 75.1 | 18.3 |
| 1 + mesosulfuron- | Lolium | F | 16 + 0.25 | 64:1 | 56.7 | 31.5 | 83.4 | 70.3 | 13.1 |
| methyl | multiflorum | | 16 + 0.5 | 32:1 | 56.7 | 44.3 | 90.6 | 75.9 | 14.7 |
| 1 + imazamox | Lolium | S | 8 + 0.4 | 20:1 | 53.2 | 25.4 | 81.4 | 65.1 | 16.3 |
| | multiflorum | | 8 + 0.8 | 10:1 | 53.2 | 42.7 | 90.3 | 73.2 | 17.1 |
| 1 + nicosulfuron | Digitaria | F | 16 + 1 | 16:1 | 42.3 | 29.7 | 80.3 | 59.4 | 20.9 |
| | sanguinalis | | 16 + 2 | 8:1 | 42.3 | 47.6 | 89.7 | 69.8 | 19.9 |
| 1 + imazethapyr | Echinochloa | S | 8 + 1 | 8:1 | 50.7 | 33.4 | 81.6 | 67.2 | 14.4 |
| | crusgalli | | 8 + 2 | 4:1 | 50.7 | 43.8 | 90.5 | 72.3 | 18.2 |
| 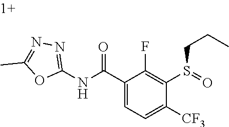 [SMILES: CCC[S@@](=O)c1c(C(F)(F)F)ccc(C(=O)Nc2nnc(C)o2)c1F] | Echinochloa crusgalli | F | 16 + 1.5 16 + 3 | 32:3 16:3 | 46.2 46.2 | 33.8 49.7 | 81.6 93.4 | 64.4 72.9 | 17.2 20.5 |
| 1 + benzobicyclon | Echinochloa | S | 8 + 2 | 4:1 | 50.7 | 22.6 | 81.4 | 61.8 | 19.6 |
| | crusgalli | | 8 + 4 | 2:1 | 50.7 | 42.8 | 90.2 | 71.8 | 18.4 |
| 1 + tefuryltrione | Echinochloa | S | 8 + 0.6 | 40:3 | 50.7 | 28.4 | 83.2 | 64.7 | 18.5 |
| | crusgalli | | 8 + 1.2 | 20:3 | 50.7 | 49.7 | 90.1 | 75.2 | 14.9 |
| 1 + fenpyrazone | Echinochloa | F | 16 + 1.5 | 32:3 | 46.2 | 32.6 | 82.1 | 63.7 | 18.4 |
| | crusgalli | | 16 + 3 | 16:3 | 46.2 | 50.7 | 90.6 | 73.5 | 17.1 |
| 1 + bipyrazone | Galium | F | 16 + 0.75 | 64:3 | 55.4 | 33.6 | 83.9 | 70.4 | 13.5 |
| | aparine | | 16 + 1.5 | 32:3 | 55.4 | 46.2 | 92.4 | 76.0 | 16.4 |
| 1 + cypyrafluone | Lolium | F | 16 + 3 | 16:3 | 56.7 | 26.7 | 86.4 | 68.3 | 18.1 |
| | multiflorum | | 16 + 6 | 8:3 | 56.7 | 38.9 | 92.2 | 73.5 | 18.7 |
| 1 + tripyrasulfone | Echinochloa | F | 16 + 4 | 4:1 | 46.2 | 26.7 | 81.5 | 60.6 | 20.9 |
| | crusgalli | | 16 + 8 | 2:1 | 46.2 | 46.1 | 90.4 | 71.0 | 19.4 |
| 1 + isoxaflutole | Echinochloa | S | 8 + 1 | 8:1 | 50.7 | 26.4 | 83.4 | 63.7 | 19.7 |
| | crusgalli | | 8 + 2 | 4:1 | 50.7 | 44.3 | 92.4 | 72.5 | 19.9 |
| 1 + mesotrione | Digitaria | F | 16 + 2.5 | 32:5 | 42.3 | 38.6 | 80.6 | 64.6 | 16.0 |
| | sanguinalis | | 16 + 5 | 16:5 | 42.3 | 52.1 | 91.7 | 72.4 | 19.3 |
| 1 + fenquinotrione | Lolium | S | 8 + 1.5 | 16:3 | 53.2 | 28.6 | 81.6 | 66.6 | 15.0 |
| | multiflorum | | 8 + 3 | 8:3 | 53.2 | 44.1 | 93.4 | 73.8 | 19.6 |
| 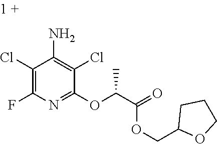 [SMILES: C[C@@H](Oc1nc(F)c(Cl)c(N)c1Cl)C(=O)OCC1CCCO1] | Lolium multiflorum | F | 16 + 8 16 + 16 | 2:1 1:1 | 56.7 56.7 | 30.7 44.1 | 85.7 94.2 | 70.0 75.8 | 15.7 18.4 |
| 1 + florpyrauxifen | Echinochloa | F | 16 + 0.5 | 32:1 | 46.2 | 22.5 | 83.7 | 58.3 | 25.4 |
| | crusgalli | | 16 + 1 | 16:1 | 46.2 | 41.3 | 90.3 | 68.4 | 21.9 |
| 1 + halauxifen | Galium | F | 16 + 0.2 | 80:1 | 55.4 | 26.4 | 83.6 | 67.2 | 16.4 |
| | aparine | | 16 + 0.4 | 40:1 | 55.4 | 37.2 | 91.7 | 72.0 | 19.7 |
| 1 + fluroxypyr | Galium | F | 16 + 4 | 4:1 | 55.4 | 29.4 | 83.3 | 68.5 | 14.8 |
| | aparine | | 16 + 8 | 2:1 | 55.4 | 46.1 | 93.2 | 76.0 | 17.2 |
| 1 + MCPA | Galium | F | 16 + 20 | 4:5 | 55.4 | 33.1 | 85.4 | 70.2 | 15.2 |
| | aparine | | 16 + 40 | 2:5 | 55.4 | 46.7 | 92.2 | 76.2 | 16.0 |
| 1 + dicamba | Galium | F | 16 + 4 | 4:1 | 55.4 | 28.6 | 85.4 | 68.2 | 17.2 |
| | aparine | | 16 + 8 | 2:1 | 55.4 | 39.7 | 92.7 | 73.1 | 19.6 |
| 1 + 2,4-D | Veronica | S | 8 + 6 | 4:3 | 48.9 | 33.6 | 84.3 | 66.1 | 18.2 |
| isooctyl ester | didyma | | 8 + 12 | 1:2 | 48.9 | 42.5 | 90.3 | 70.6 | 19.7 |
| 1 + aclonifen | Lolium | S | 8 + 20 | 2:5 | 53.2 | 31.6 | 88.4 | 68.0 | 20.4 |
| | multiflorum | | 8 + 40 | 1:5 | 53.2 | 52.1 | 94.7 | 77.6 | 17.1 |
| 1 + cinmethylin | Echinochloa | S | 8 + 0.4 | 20:1 | 50.7 | 26.4 | 76.8 | 63.7 | 13.1 |
| | crusgalli | | 8 + 0.8 | 10:1 | 50.7 | 38.6 | 89.6 | 69.7 | 19.9 |
| 1 + clomazone | Echinochloa | S | 8 + 2 | 4:1 | 50.7 | 33.2 | 80.3 | 67.1 | 13.2 |
| | crusgalli | | 8 + 4 | 2:1 | 50.7 | 46.8 | 93.4 | 73.8 | 19.6 |
| 1 + bixlozone | Echinochloa | S | 8 + 2 | 4:1 | 50.7 | 41.3 | 87.2 | 71.1 | 16.1 |
| | crusgalli | | 8 + 4 | 2:1 | 50.7 | 50.3 | 93.6 | 75.5 | 18.1 |
| 1 + tetflupyrolimet | Echinochloa | S | 8 + 1 | 8:1 | 50.7 | 34.7 | 86.2 | 67.8 | 18.4 |
| | crusgalli | | 8 + 2 | 4:1 | 50.7 | 55.6 | 95.7 | 78.1 | 17.6 |
| 1 + indaziflam | Digitaria | F | 16 + 0.8 | 20:1 | 42.3 | 36.4 | 85.4 | 63.3 | 22.1 |
| | sanguinalis | | 16 + 1.6 | 10:1 | 42.3 | 52.1 | 90.3 | 72.4 | 17.9 |
| 1 + triaziflam | Echinochloa | S | 8 + 2 | 4:1 | 50.7 | 29.4 | 83.6 | 65.2 | 18.4 |
| | crusgalli | | 8 + 4 | 2:1 | 50.7 | 40.5 | 89.4 | 70.7 | 18.8 |
| 1 + oxaziclomefone | Echinochloa | S | 8 + 0.5 | 16:1 | 50.7 | 33.7 | 79.5 | 67.3 | 12.2 |
| | crusgalli | | 8 + 1 | 8:1 | 50.7 | 51.2 | 90.2 | 75.9 | 14.3 |

At the same time, it has been found after many tests that many of the compounds and compositions thereof of the present invention have good selectivity to many grammneae grasses such as Zoysia japonica, bermuda grass, tall fescue, bluegrass, ryegrass and seashore paspalum etc, and are able to control many important grass weeds and broad-leaved weeds. The compounds also show excellent selectivity and commercial values in the tests on sugarcane, oil sunflower, potato, fruit trees and vegetables in different herbicide application methods.

1. A 3-isoxazolidinone compound, as shown in Formula I: 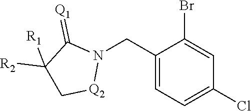
wherein, Q₁ and Q₂ each independently represent O or S;
R₁ and R₂ each independently represent alkyl, preferably C1-C8 alkyl, more preferably C1-C6 alkyl.

2. The 3-isoxazolidinone compound according to claim 1, characterized in that,
Q₁ and Q₂ each independently represent O;
R₁ and R₂ each independently represent methyl.

3. A method for preparing the 3-isoxazolidinone compound according to claim 1, characterized in that comprising the following steps:
subjecting the compound of the general formula II and the compound of the general formula III to substitution reaction to obtain the compound of the general formula I, and the chemical reaction equation is as follows: 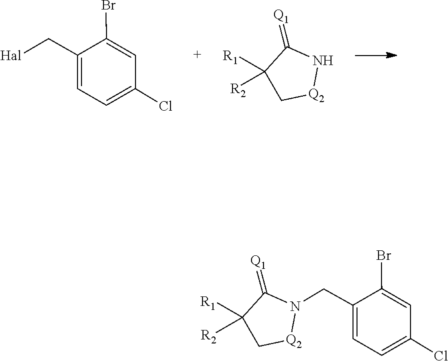
wherein, Hal represents halogen; other substituents Q₁, Q₂, R₁ and R₂ are as described in claim 1;
preferably, the reaction is carried out in the presence of a base and a solvent; more preferably, the base is selected from at least one of inorganic bases or organic bases; more preferably, the solvent is selected from at least one of DMF, methanol, ethanol, acetonitrile, dichloroethane, DMSO, Dioxane, dichloromethane and ethyl acetate.

4. A herbicidal composition, characterized in that comprising (i) a herbicidally effective amount of at least one of the 3-isoxazolidinone compounds according to claim 1.

5. The herbicidal composition according to claim 4, characterized in that further comprising (ii) a herbicidally effective amount of one or more additional herbicides and/or safeners.

6. The herbicidal composition according to claim 5, characterized in that the additional herbicide is selected from one or more of the following compounds: mesotrione, tefuryltrione, benzobicyclon, bipyrazone, cypyrafluone, tripyrasulfone, fenpyrazone, fenquinotrione, 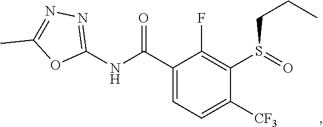 [SMILES: CCC[S@@](=O)c1c(C(F)(F)F)ccc(C(=O)Nc2nnc(C)o2)c1F]
isoxaflutole, flurochloridone, diflufenican, picolinafen, beflubutamid, fluridone, clomazone, bixlozone, bensulfuron-methyl, pyrazosulfuron-ethyl, halosulfuron, propyrisulfuron, metazosulfuron, ethoxysulfuron, mesosulfuron-methyl, nicosulfuron, triafamone, penoxsulam, imazamox, imazethapyr, florasulam, bispyribac, oxyfluorfen, fomesafen, oxadiazon, oxadiargyl, sulfentrazone, pyraclonil, flumioxazin, pentoxazone, tiafenacil, saflufenacil, trifludimoxazin, epyrifenacil, 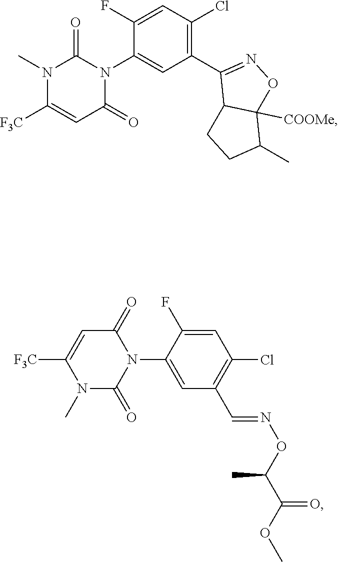 [SMILES: COC(=O)C12ON=C(c3cc(-n4c(=O)cc(C(F)(F)F)n(C)c4=O)c(F)cc3Cl)C1CCC2C ; COC(=O)[C@@H](C)O/N=C/c1cc(-n2c(=O)cc(C(F)(F)F)n(C)c2=O)c(F)cc1Cl]
benmicaozuo, atrazine, prometryne, simetryn, terbutylazine, terbutryne, ametryne, metribuzin, amicarbazone, chlorotoluron, isoproturon, diuron, propanil, bentazon, bromoxynil octanoate, butralin, pendimethalin, dithiopyr, acetochlor, butachlor, pretilachlor, metolachlor, mefenacet, S-metolachlor, flufenacet, napropamide, pyroxasulfone, anilofos, dimethenamid, fentrazamide, ipfencarbazone, dimethenamid-P, bromobutide, prosulfocarb, molinate, quinclorac, fluroxypyr, fluroxypyr-meptyl, halauxifen, dicamba, florpyrauxifen, (4-chloro-2-methylphenoxy)acetic acid, 2-methyl-4-chlorophenoxymonothioacetic acid S-ethyl ester, 2-methyl-4-chlorophenoxyacetic acid sodium salt, (4-chloro-2-methylphenoxy)acetic acid 2-ethylhexyl ester, 2,4-dichlorophenoxyacetic acid butyl ester, 2,4-dichlorophenoxyacetic acid isooctyl ester, 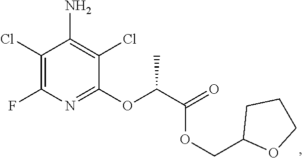 [SMILES: C[C@@H](Oc1nc(F)c(Cl)c(N)c1Cl)C(=O)OCC1CCCO1]
(2,4-dichlorophenoxy)acetic acid, (2-methyl-4-chlorophenoxy)acetic acid dimethylamine salt, triaziflam, indaziflam, aclonifen, cinmethylin, tetflupyrolimet, and oxaziclomefone.

7. The herbicidal composition according to claim 6, characterized in that the active ingredient (i) to the additional herbicide in (ii) in the herbicidal composition is in a weight ratio of 1:1000˜1000:1, 1:800˜800:1, 1:600˜600:1, 1:500˜500:1, 1:400˜400:1, 1:300˜300:1, 1:200˜200:1, 1:180˜180:1, 1:150˜150:1, 1:120˜120:1, 1:100˜100:1, 1:80˜80:1, 1:50˜50:1, 1:30˜30:1, 1:20˜20:1, 1:10˜10:1, 1:5˜1:1 or 1:1˜5:1.

8. The herbicidal composition according to claim 4, characterized in that further comprising (iii) an agrochemically acceptable formulation auxiliary.

9. A method for controlling an undesirable plant, characterized in that comprising applying at least one of the 3-isoxazolidinone compounds according to claim 1 in a herbicidally effective amount on a plant or in its area or to soil or water to control the emergence or growth of an undesirable plant; preferably, the undesirable plant includes herbicide-resistant or -tolerant weed species.

10. A use of at least one of the 3-isoxazolidinone compounds according to claim 1 for controlling an undesirable plant; preferably, the 3-isoxazolidinone compound is used to control a weed among a useful crop; more preferably, the useful crop includes a transgenic crop or a crop treated by genome editing techniques, and the weed includes herbicide-resistant or -tolerant weed species.
